(19) United States
(12) Patent Application Publication
(10) Pub. No.: US 2015/0004190 A1
(43) Pub. Date: Jan. 1, 2015

(54) VACCINE COMPOSITIONS FOR HIV PREVENTION AND TREATMENT

(71) Applicants: Institut National De La Sante Et De La Recherche Medicale (INSERM), Paris (FR); Universite Paris Descartes, Paris (FR); Centre National de la Recherche Scientifique (C.N.R.S.), Paris (FR); Universite Paris Diderot - Paris 7, Paris (FR)

(72) Inventors: Morgane Bomsel, Paris (FR); Daniela Tudor, Paris (FR)

(21) Appl. No.: 14/374,121

(22) PCT Filed: Jan. 25, 2013
(86) PCT No.: PCT/EP2013/051503

§ 371 Date: Jul. 23, 2014

(30) Foreign Application Priority Data
Jan. 26, 2012 (EP) 12305098.1

(51) Int. Cl. A61K 39/21; C12N 7/00

(52) U.S. Cl. CPC A61K 39/21; C12N 7/00; C12N 2740/16034
USPC 424/188.1; 435/5; 530/327; 530/389.4; 536/23.72

(57) ABSTRACT

Novel antibodies and peptides relating to HIV are disclosed, as are vaccine compositions containing such antibodies and peptides. The compounds and compositions of the invention are of use in prevention and treatment of HIV, and in particular for inducing mucosal immunity to HIV.

### FIELD OF THE INVENTION

Novel antibodies and peptides relating to HIV are disclosed, as are vaccine compositions containing such antibodies and peptides. The compounds and compositions of the invention are of use in prevention and treatment of HIV, and in particular for inducing mucosal immunity to HIV.

### BACKGROUND OF THE INVENTION

Human immunodeficiency virus (HIV-1) is transmitted mainly sexually. The mucosal surfaces of the genital and gastrointestinal tract are thus the principal sites of entry for HIV infection. Establishing selective protection against HIV infection at these sites, either by inducing a mucosal HIV-specific response by a vaccine or by characterising specific antiviral agents for inclusion in microbicide formulations, is thus a potentially promising but so far elusive treatment goal.

A prophylactic vaccine would preferably focus on the induction of a protective immunity at mucosal surfaces involving both IgA and IgG specific for HIV-1, as IgA and IgG have different but complementary antiviral functions. IgG induces efficient antibody dependent cellular cytotoxicity (ADCC) and neutralizes infection of target CD4+ T cells, while IgA blocks HIV transcytosis or HIV-1 entry in dendritic cells and can exert neutralizing activities against CD4+ T cell infection.

The role of gp41-specific IgA in protection against sexual transmission of HIV has been exemplified in highly HIV-1 exposed but persistently IgG seronegative (HEPS) individuals. Analysis of their mucosal immune response revealed the presence of gp41-specific IgA as a correlate to protection in the absence of HIV-1 specific IgGs. Such IgAs could neutralize CD4+ T cell infection and block HIV-1 transcytosis. Importantly, in contrast to IgGs, mucosal IgAs are more resistant to enzymatic degradation in the harsh genital milieu, and lack inflammatory properties as they do not result in the activation of the complement system. Complement activation is followed by the local tissue damage and an increased absorption of bystander antigens.

According to the current paradigm, the antibody molecule is composed of two non-interacting and largely independent domains: (i) the variable (V) domain that confers antigen specificity and affinity and (ii) the heavy chain constant (CH)-domains that dictate its effector functions. This implies that the CH-region does not participate in antigen binding, and that isotype switching changes the antibody effector's function without changing its specificity.

This paradigm was recently challenged by studies showing that isotype switching contributes to antibody affinity, via the avidity factor. Furthermore, antibody specificity is not solely the result of (V)-region interactions with an antigen. The (CH)-domains can affect affinity and fine specificity, as well as V-region structure. Thus, the antibody molecule appears now as a whole entity with regards to its binding, and effector functions.

The apparent influence of the CH-region on affinity and fine specificity suggests that the quality of the secondary/memory immune response, stronger or weaker, may also depend on the antibody isotype.

In humans, four main IgG isotypes occur, namely IgG1, IgG2, IgG3, and IgG4 differing in the length and flexibility of the hinge region. The flexibility of the hinge region decreases in the following order: IgG3>IgG1>IgG4>IgG2, that probably relates to the higher activity of IgG3 in triggering effector functions, when compared to the other subclasses. In the case of HIV-1 infection, the dominant isotype is IgG1, while IgG2, IgG3, and IgG4 are less or rarely produced, although the broadly neutralizing IgG2F5 was originally isolated as an IgG3. IgG3 has an extended hinge region that provides spacing and flexibility. This facilitates simultaneous antigen and Fc-R binding, and enhances the potential for high affinity functional interactions between effectors and cell surface or structurally distant antigens.

IgA exists as two subclasses, IgA1 and IgA2, found in similar amounts in the genital tract but not in the serum, the respiratory tract and upper intestinal tract, where IgA1 largely dominates. Three IgA2 allotypes, IgA2m(1), IgA2m(2) and IgA2(n) were described in humans, which differ in their H and L chain disulfide binding patterns. Whereas in the cervical mucus IgA is rather polymeric and associated to J chain, in the vagina equal proportions of monomeric and dimeric IgA are found, suggesting that monomeric IgA may also play a role in protection against incoming pathogens at this site. The two IgA subclasses appeared by gene duplication and thus share many similarities, the major difference being the hinge region length that account for their different susceptibilities to proteases secreted by many important mucosal pathogens. The 23 residues with 10 interspaced prolines of IgA1 hinge region is glycosylated and the target of IgA1 protease, in contrast to the 10 residues of IgA2m(1) hinge region containing 6 contiguous prolines. Consequently, monomeric IgA2m(1) is more rigidly hinged and compact with Fab to Fab distance of around 8 nm than monomeric IgA1 whose Fab to Fab distance is 17 nm and less susceptible to degradation. Additionally, IgA2(m)1 light chains can be disulphide-bridged together by their C termini reducing overall antibody flexibility as well. The reduced flexibility of IgA2(m1) is however enough to enable the two Fab fragments to adopt a wide range of optimal antigen-binding conformations, while ensuring their maintenance in an extended conformation relative to the Fc fragment. The differences between IgA1 and IgA2 could account for their biological distinct properties and their functional activity. Both subclasses have reduced hinge flexibilities compared to IgG, whose non-glycosylated hinge regions contain few prolines but glycines also that provide flexibility.

In earlier studies concerning the biological effect of constant heavy chain in HIV binding and neutralization (Liu, Bergami et al. 2003), a panel of antibodies was created by switching the IgG2 human monoclonal antibody F425B4e8F, specific for the base of the hyper variable V3 loop of the HIV-1 envelope gpl20. The resulting IgA captured significantly more virus than the corresponding IgG2, IgG3 and IgG1, with the greatest binding occurring for both infected cells, and dual tropic R5X4 cell free virus. However, no statistical differences in the neutralization between various isotypes were observed (Liu, Bergami et al. 2003).

Two other broadly neutralizing anti-HIV-1 human Abs, 2F5 and 2G12 were also previously class switched from IgG1 to IgM and IgA. 2F5 dimeric-IgA1 and 2G12 IgM applied at the serosal (basal) side of a tight epithelium block transcytosis of HIV-1 in a plgR-mediated manner (Wolbank, Kunert et al. 2003), in contrast to the corresponding IgGs that are unable to bind the plgR. In these experiments, HIV-1 transcytosis block from the mucosal side was not tested. Furthermore, 2F5 dimeric-IgA1 was also poor at blocking HIV transcytosis across an epithelial monolayer as well as HIV-1 translocation across a rectal tissue (Shen, Drelichman et al. 2010). Finally, switching the broadly neutralizing IgG1 b12 specific for gpl20 to monomeric, dimeric and polymeric IgA2s results in IgA with rather poor neutralizing activities against R5 and X4 tropic HIV-1 infection of CD4+ cells, and with an equally potential as IgG1 to block HIV-1 uptake by epithelial cells (Mantis, Palaia et al. 2007). None of these studies took into account the potential influence on antibody affinity and functional activity of the IgA isotype, namely 1 versus 2, with their characteristic CH1 domain.

The inventors thus evaluated the respective role of antibody isotype and CH1 domain relative to their functional activities at the molecular level using the broadly neutralizing human mAb 2F5, directed against the membrane proximal region (MPER) of the HIV-1 envelope transmembrane subunit gp41. They have shown that the CH1 region can clearly affect the antibody specificity and affinity and is determinant in the magnitude of the antibody functional activities, as described below.

### DESCRIPTION OF THE INVENTION

The inventors have created IgA2 and IgG1 isotypes carrying the epitope-binding domains of the neutralising anti-HIV-1 antibody 2F5, which was originally isolated as an IgG3, and investigated the role of the antibody CH1 constant region on epitope specificity and antiviral activities. They established that the IgA2 CH1 region plays an important role in all of these activities, resulting in activities specific for the IgA2 isoform. Analysis of the epitopes bound by the IgA2 and IgG1 antibodies showed that different epitopes are recognised by the different isoforms, which recognise a substantially different 3D conformational epitope on gp41. Using epitopes specific for each isotype to induce production of IgA and/or IgG antibodies could thus be a promising strategy for HIV vaccination, enabling optimal mucosal immunity to be achieved by stimulation of production of IgA2. The 2F5 IgA2 antibody itself also represents a promising therapeutic agent for conferring mucosal immunity to HIV-1.

Thus, not only have the inventors for the first time created an IgA2 isotype of the 2F5 antibody, they have for the first time shown that the isotype and in particular the CH1 constant region can alter 2F5 binding specificity. The IgA2 and IgG1 isotopes of 2F5 were shown to bind to different conformational epitopes. Short peptides mimicking these eptiopes, which represent non-conguous sequence from within gp41, are shown in FIGS. 9 and 10 (SEQ ID NOs 1-47).

In brief, the role of antibody CH1 constant region on epitope specificity, affinity, and antiviral activities was investigated using the 2F5 IgG1, one of the rare anti-HIV-1 human antibody specific for the HIV-1 viral envelope glycoprotein gp41 with broadly neutralizing activities, isotype-switched to an IgA2. 2F5 as IgA2 bound to gp41 and P1, an extended gp41-membrane proximal region inserted in a lipidic membrane with higher affinities than IgG1. Functionally, 2F5 IgA2 blocked more efficiently than IgG1 the HIV-1 transcytosis across epithelial cells and CD4+ infection cells by R5 HIV-1. Whereas neither 2F5 IgG1, nor IgA2 blocked efficiently HIV-1 transfer from Langerhans cells to autologous CD4+ T cells, both isotypes acted synergistically in blocking virus transfer and also T cell infection. Epitope mapping performed by screening a random peptide library and docking modeling in silico showed that, in addition to the 2F5 IgG canonical ELDKWA epitope on gp41, the IgG1 recognized an additional epitope on the gp41 C-helix, whereas the IgA2 epitope included a novel motif on the gp41 N-helix also. Altogether, the CH1 region appeared to fine-tune the specificity of variable-regions of identical antibodies by modulating epitope specificity, antibody affinity and functional activities. In the context of sexually transmitted infections such as HIV-1/AIDS, raising a mucosal IgA-based vaccine response should complement an IgG one in blocking HIV-1 transmission.

Thus, in one aspect the invention provides a peptide comprising or consisting of the consecutive sequence of amino acids of a sequence shown in FIG. 9 (SEQ ID Nos 1-22) or FIG. 10 (SEQ ID Nos 23-47).

In another aspect, the invention provides an IgA2 antibody binding to HIV-1 gp41. Preferably, the antibody is a human antibody. In preferred embodiments, the antibody comprises the CDR sequences shown in SEQ ID Nos 50-55. In some embodiments, the antibody comprises the variable regions of monoclonal antibody 2F5 as described in Kunert, Ruker et al. (1998) AIDS Res Hum Retroviruses 14(13): 1115-28). In some embodiments, the antibody comprises the constant region of human IgA2 as described in Berdoz, Blanc et al. (1999) Proc Natl Acad Sci USA 96(6): 3029-34). In a particularly preferred embodiment, the antibody has a VH chain comprising the sequence of SEQ ID No 56 and/or a VL chain comprising the sequence of SEQ ID No 57.

In one embodiment, an antibody of the invention as a heavy chain comprising one or more of the following CDR sequences:

VH CDR1
(SEQ ID No 50)
GFSLSDFGVG;
VH CDR2
(SEQ ID No 51)
IYSDDDK;
and
VH CDR3
(SEQ ID No 52)
AHRRGPTTLFGVPIARGPVNAMDV;

and/or a light chain comprising one or more of the following CDR sequences

VL CDR1
(SEQ ID No 53)
QGVTSA;
VL CDR2
(SEQ ID No 54)
DAS;
and
VL CDR3
(SEQ ID No 55)
QQLHFYPHT;

and optionally a heavy chain comprising the amino acid sequence:

(SEQ ID No 56)
RITLKESGP.PLVKPTQTLTLTCSFSGFSLSDFGVGVGWIRQPPGKA
LEWLAIIYSDDDKRYSPSLN.TRLTITKDTSKNQVVLVMTRVSPVDT
ATYFCAHRRGPTTLFGVPIARGPVNAMDVWGQGITVTI

and/or a light chain comprising the amino acid sequence:

(SEQ ID No 57)
ALQLTQSPSSLSASVGDRITITCRASQGVTSALAWYRQKPGSPPQLLI
YDASSLESGVPSRFSGSGSGTEFTLTISTLRPEDFATYYCQQLHFYPH
TFGGGTRVDVR.

The antibody of the invention may be an IgA2 or an IgG1.

Also provided are nucleic acids encoding the peptides and antibodies of the invention, vectors comprising said nucleic acids and host cells comprising said vectors and nucleic acids.

Also provided is a composition comprising a peptide, antibody or nucleic acid of the invention. In a preferred embodiment, said composition is a vaccine composition. In some embodiments, said composition is suitable for mucosal administration. For example, said composition may be suitable for administration to the nasal, rectal or vaginal mucosa. In some embodiments, said vaccine composition additionally comprises one or more adjuvants.

In a preferred embodiment, said composition comprises at least one peptide of FIG. 9 (SEQ ID Nos 1-22) and/or at least one peptide of FIG. 10 (SEQ ID Nos 23-47), or nucleic acids encoding said peptide.

Preferably, said composition comprises a peptide of one of more of SEQ ID Nos 8, 9, 10, 32, 34, 35, 36, 37 and 40, or a nucleic acid encoding said peptide.

Optionally, the composition is administered in a prime boost regimen. The prime boost regimen may comprise administration of gp41 or a nucleic acids encoding gp41. An exemplary form of gp41 is shown in SEQ ID Nos 48 and 49 (helices 1 and 2 of gp41 protein) having the following sequences:

gp41 helix 1:
DDDDKMEWDREYNNYTSLIHSLIEESQNQQEKNEQELLELDKWAS
LWNWFNNWLWYIT
gp41 helix 2:
GAASMTLTVQARQLLSGIVQQQNNLLRAIEAQQHLLQLTVWGIKQ
LQARIL

Also provided is a peptide, antibody, nucleic acid or composition of the invention for use in the preventive treatment of HIV or AIDS. The composition may be for administration to a mucosal surface, or any other administrative route such as those described below. The composition may be for administration in a prime-boost regimen such as those described below, such as a regimen comprising administration of full-length HIV-1 gp41.

Also provided is a peptide, nucleic acid or composition of the invention for use in a method of inducing a mucosal immune response to HIV-1. In some embodiments, said method comprises administering said peptide, nucleic acid or composition to a subject, in particular a subject in need thereof. The peptide, nucleic acid or composition may be administered to a mucosal surface of a subject, for example the nasal, rectal or vaginal mucosa. Various administration regimens are described herein and encompassed within the scope of the invention.

Also provided is a peptide of FIG. 9 (SEQ ID Nos 1-22), a nucleic acid encoding said peptide or composition comprising said peptide for use in a method of inducing a mucosal IgA2 response to HIV-1.

Also provided is a peptide of FIG. 10 (SEQ ID Nos 23-47) a nucleic acid encoding said peptide or composition comprising said peptide for use in a method of inducing a mucosal IgG1 response to HIV-1.

Also provided is a peptide, nucleic acid or composition of the invention for use in a method of inducing a mixed mucosal IgG1 and IgA2 response to HIV-1, the method comprising administration of at least one peptide of FIG. 9 (SEQ ID Nos 1-22) and at least one peptide of FIG. 10 (SEQ ID Nos 23-47), or nucleic acids encoding said peptides, or a composition comprising said peptides or nucleic acids.

Also provided is the use of a peptide, antibody, nucleic acid or composition of the invention for use in the preparation of a medicament for the preventive treatment of HIV infection, in particular HIV-1 infection, or AIDS.

Also provided is a method of prevention or treatment of HIV infection, in particular HIV-1 infection, comprising administration of a composition of the invention to an individual in need thereof. In some embodiments, said composition is administered to a mucosal surface of a subject, for example the nasal, rectal or vaginal mucosa. Various administration regimens are described herein and encompassed within the scope of the invention.

Also provided is a method of inducing a mucosal immune response to HIV-1, comprising prevention or treatment of HIV infection, comprising administration of a composition of the invention to an individual in need thereof. In some embodiments, said composition is administered to a mucosal surface of a subject, for example the nasal, rectal or vaginal mucosa. Various administration regimens are described herein and encompassed within the scope of the invention.

Also provided is a method of inducing a mucosal IgA2 response to HIV-1, the method comprising administering at least one peptide of FIG. 9 (SEQ ID Nos 1-22) or a nucleic acid encoding said peptide, or a composition comprising said peptide or nucleic acid, to an individual in need thereof, as described herein.

Also provided is a method of inducing a mucosal IgG1 response to HIV-1, the method comprising administering at least one peptide of FIG. 10 (SEQ ID Nos 23-47) or a nucleic acid encoding said peptide, or a composition comprising said peptide or nucleic acid, to an individual in need thereof, as described herein.

Also provided is a method of inducing a mixed mucosal IgG1 and IgA2 response to HIV-1, the method comprising administering at least one peptide of FIG. 9 (SEQ ID Nos 1-22) and at least one peptide of FIG. 10 (SEQ ID Nos 23-47) or nucleic acids encoding said peptides, or a composition comprising said peptide or nucleic acid to an individual in need thereof, as described herein.

Also provided is method of diagnosis of HIV infection or AIDS, said method comprising detection of a gp41 protein in a biological sample from a patient using an antibody of the invention. The biological sample may be a body fluid from a subject, for example, blood, plasma, serum, semen, vaginal discharge or urine.

### HIV/AIDS

AIDS, or Acquired Immunodeficiency Syndrome, is caused by human immunodeficiency virus (HIV) and is characterised by several clinical features including wasting syndromes, central nervous system degeneration and profound immunosuppression that results in opportunistic infections and malignancies. HIV is a member of the lentivirus family of animal retroviruses, which include the visna virus of sheep and the bovine, feline, and simian immunodeficiency viruses (SIV). Two closely related types of HIV, designated HIV-1 and HIV-2, have been identified thus far, of which HIV-1 is by far the most common cause of AIDS. However, HIV-2, which differs in genomic structure and antigenicity, causes a similar clinical syndrome.

An infectious HIV particle consists of two identical strands of RNA, each approximately 9.2 kb long, packaged within a core of viral proteins. This core structure is surrounded by a phospholipid bilayer envelope derived from the host cell membrane that also includes virally-encoded membrane proteins. The HIV genome has the characteristic 5′-LTR-Gag-Pol-Env-LTR-3′ organization of the retrovirus family. Long terminal repeats (LTRs) at each end of the viral genome serve as binding sites for transcriptional regulatory proteins from the host and regulate viral integration into the host genome, viral gene expression, and viral replication.

The HIV genome encodes several structural proteins. The gag gene encodes structural proteins of the nucleocapsid core and matrix. The pol gene encodes reverse transcriptase (RT), integrase (IN), and viral protease (PR) enzymes required for viral replication. The tat gene encodes a protein that is required for elongation of viral transcripts. The rev gene encodes a protein that promotes the nuclear export of incompletely spliced or unspliced viral RNAs. The vif gene product enhances the infectivity of viral particles. The vpr gene product promotes the nuclear import of viral DNA and regulates G2 cell cycle arrest. The vpu and nef genes encode proteins that down regulate host cell CD4 expression and enhance release of virus from infected cells. The env gene encodes the viral envelope glycoprotein that is translated as a 160-kilodalton (kDa) precursor (gpl60) and cleaved by a cellular protease to yield the external 120-kDa envelope glycoprotein (gpl20) and the transmembrane 41-kDa envelope glycoprotein (gp41), which are required for the infection of cells (Abbas et al., Cellular and Molecular Immunology, 4th edition, W.B. Saunders Company, 2000, pp. 454-456). gpl40 is a modified form of the Env glycoprotein, which contains the external 120-kDa envelope glycoprotein portion and the extracellular part of the gp41 portion of Env and has characteristics of both gpl20 and gp41. The nef gene is conserved among primate lentiviruses and is one of the first viral genes that is transcribed following infection. In vitro, several functions have been described, including downregulation of CD4 and MHC class I surface expression, altered T-cell signaling and activation, and enhanced viral infectivity.

HIV infection initiates with gpl20 on the viral particle binding to the CD4 and chemokine receptor molecules (e.g., CXCR4, CCR5) on the cell membrane of target cells such as CD4⁺ T-cells, macrophages and dendritic cells. The bound virus fuses with the target cell and reverse transcribes the RNA genome. The resulting viral DNA integrates into the cellular genome, where it directs the production of new viral RNA, and thereby viral proteins and new virions. These virions bud from the infected cell membrane and establish productive infections in other cells. This process also kills the originally infected cell. HIV can also kill cells indirectly because the CD4 receptor on uninfected T-cells has a strong affinity for gpl20 expressed on the surface of infected cells. In this case, the uninfected cells bind, via the CD4 receptor-gpl20 interaction, to infected cells and fuse to form a syncytium, which cannot survive. Destruction of CD4⁺ T-lymphocytes, which are critical to immune defense, is a major cause of the progressive immune dysfunction that is the hallmark of AIDS disease progression. The loss of CD4⁺ T cells seriously impairs the body's ability to fight most invaders, but it has a particularly severe impact on the defenses against viruses, fungi, parasites and certain bacteria, including mycobacteria.

Gp41 is the most conserved envelope subunit of HIV-1 with very few potential glycosylation sites, unlike the surface envelope subunit gpl20 whose neutralising epitopes are widely masked by glycans. Gp41 thus represents a promising vaccine target. Previous work by the inventors has shown that an HIV-1 vaccine made of gp41-subunit antigens grafted on virosomes can confer immunity against mucosal HIV-1 challenge, following immunisation via both intramuscular and intranasal routes (Bomsel et al, 2011, Immunity 34: 269-280).

### Peptides

The invention relates in part to novel peptides, as described above. ‘Peptide’ refers to any peptide comprising two or more amino acids joined to each other by peptide bonds or modified peptide bonds, i.e., peptide isosteres. Polypeptide refers to both short chains, commonly referred to as peptides, oligopeptides or oligomers, and to longer chains, generally referred to as proteins. Polypeptides may contain amino acids other than the 20 gene-encoded amino acids.

Polypeptides include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini, it will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from natural posttxans!ational processes or may be made by synthetic methods. Modifications include acetylation, acylation, ADP-ribosylation, araidation, covalent attachment of flavin, covalent attachment of a heme moiety, covalent attachment of a nucleotide or nucleotide derivative, covalent attachment of a lipid or lipid derivative, covalent attachment of phosphatidyl inositol, cross-linking, cyclization, disulfide bond formation, demethylation, formation of covalent cross-links, formation of cystine, formation of pyroglutarnate, formylation, gamma-carboxy!ation, glycosylation, GPI anchor formation, hydroxy! ation, iodination, methylation, myristoylation, oxidation, proteolytic processing, phosphorylation, prenylation, racemizaiion, selenoyiation, sulfation, transfer-RNA mediated addition of amino acids to proteins such as arginyiation, and ubiquitination.

### Antibodies

The invention further provides antibodies which bind to HIV-1 gp41, as described above. the term ‘antibody’ is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term ‘monoclonal antibody’ as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier ‘monoclonal’ indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier ‘monoclonal’ indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al, Hybridoma, 14 (3): 253-260 (1995), Harlow et al, Antibodies: A Laboratory Manual, (Cold Spring Harbor Laboratory Press, 2nd ed. 1988); Hammerling et al, in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al, Proc. Natl. Acad. Sci. USA 90: 2551 (1993); Jakobovits et ah, Nature 362: 255-258 (1993); Bruggemann et ah, Year in Immunol. 7:33 (1993); U.S. Pat. Nos. 5,545,807; 5,545,806; 5,569,825; 5,625,126; 5,633,425; and 5,661,016; Marks et al, Bio/Technology 10: 779-783 (1992); Lonberg et al., Nature 368: 856-859 (1994); Morrison, Nature 368: 812-813 (1994); Fishwild et al, Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include ‘chimeric’ antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al, Proc. Natl. Acad. Sci. USA 81:6851-6855 (1984)). Chimeric antibodies include PRIMATIZED® antibodies wherein the antigen-binding region of the antibody is derived from an antibody produced by, e.g., immunising macaque monkeys with the antigen of interest.

Unless indicated otherwise, the expression ‘multivalent antibody’ denotes an antibody comprising three or more antigen binding sites. In certain embodiment, the multivalent antibody is engineered to have the three or more antigen binding sites and is generally not a native sequence IgM or IgA antibody.

A ‘human antibody’ is one which possesses an amino acid sequence which corresponds to that of an antibody produced by a human and/or has been made using any of the techniques for making human antibodies as disclosed herein. Human antibodies can be produced using various techniques known in the art, including phage-display libraries. Hoogenboom and Winter, J. Mol. Biol, 227:381 (1991); Marks et al., J. Mol. Biol, 222:581 (1991). Also available for the preparation of human monoclonal antibodies are methods described in Cole et al., Monoclonal Antibodies and Cancer Therapy, Alan R. Liss, p. 77 (1985); Boerner et al, J. Immunol, 147(I):86-95 (1991). See also van Dijk and van de Winkel, Curr. Opin. Pharmacol, 5: 368-74 (2001). Human antibodies can be prepared by administering the antigen to a transgenic animal that has been modified to produce such antibodies in response to antigenic challenge, but whose endogenous loci have been disabled, e.g., immunised xenomice (see, e.g., U.S. Pat. Nos. 6,075,181 and 6,150,584 regarding XENOMOUSE™ technology). See also, for example, Li et al, Proc. Natl. Acad. Sci. USA, 103:3557-3562 (2006) regarding human antibodies generated via a human B-cell hybridoma technology.

The ‘variable region’ or ‘variable domain’ of an antibody refers to the amino-terminal domains of the heavy or light chain of the antibody. The variable domain of the heavy chain may be referred to as ‘VH.’ The variable domain of the light chain may be referred to as ‘VL.’ These domains are generally the most variable parts of an antibody and contain the antigen-binding sites.

The term ‘variable’ refers to the fact that certain portions of the variable domains differ extensively in sequence among antibodies and are used in the binding and specificity of each particular antibody for its particular antigen.

However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

‘Antibody fragments’ comprise a portion of an intact antibody, preferably comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments.

‘Fv’ is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a ‘dimeric’ structure analogous to that in a two-chain Fv species. It is in this configuration that the three HVRs of each variable domain interact to define an antigen-binding site on the surface of the VH-VL dimer.

Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CHI) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term ‘hypervariable region,’ ‘HVR,’ or ‘HV,’ when used herein refers to the regions of an antibody variable domain which are hypervariable in sequence and/or form structurally defined loops. Generally, antibodies comprise six HVRs; three in the VH (HI, H2, H3), and three in the VL (LI, L2, L3). In native antibodies, H3 and L3 display the most diversity of the six HVRs, and H3 in particular is believed to play a unique role in conferring fine specificity to antibodies. See, e.g., Xu et al, Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

‘Framework’ or ‘FR’ residues are those variable domain residues other than the HVR residues as herein defined.

The term ‘Fc region’ herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A ‘functional Fc region’ possesses an ‘effector function’ of a native sequence Fc region. Exemplary ‘effector functions’ include Clq binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

A ‘native sequence Fc region’ comprises an amino acid sequence identical to the amino acid sequence of an Fc region found in nature. Native sequence human Fc regions include a native sequence human IgGI Fc region (non-A and A allotypes); native sequence human IgG2 Fc region; native sequence human IgG3 Fc region; and native sequence human IgG4 Fc region as well as naturally occurring variants thereof.

A ‘variant Fc region’ comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

‘Fc receptor’ or ‘FcR’ describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRII) subclasses, including allelic variants and alternatively spliced forms of those receptors. FcγRII receptors include FcγRIIA (an ‘activating receptor’) and FcγRIIB (an ‘inhibiting receptor’), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain, (see, e.g., Daeron, Annu. Rev. Immunol. 15:203-234 (1997)). FcRs are reviewed, for example, in Ravetch and Kinet, Annu. Rev. Immunol 9:457-92 (1991); Capel et ah, Immunomethods 4:25-34 (1994); and de Haas et ah, J. Lab. CHn. Med. 126:330-41 (1995). Other FcRs, including those to be identified in the future, are encompassed by the term ‘FcR’ herein.

### Protein and Nucleic Acid Variants

Variants of the antibodies and peptides disclosed herein, and their encoding nucleic acids, are also encompassed within the scope of the invention. Variant proteins may be naturally occurring variants, such as splice variants, alleles and isoforms, or they may be produced by recombinant means. Variations in amino acid sequence may be introduced by substitution, deletion or insertion of one or more codons into the nucleic acid sequence encoding the protein that results in a change in the amino acid sequence of the protein. Optionally the variation is by substitution of 1, 2, 3, 4, 5, 6, 7, 8, 9, 10, 11, 12, 13, 14, 15, 16, 17, 18, 19, 20 or more amino acids with any other amino acid in the protein. Amino acid substitutions may be conservative or non-conservative. Preferably, substitutions are conservative substitutions, in which one amino acid is substituted for another amino acid with similar structural and/or chemical properties. Additionally or alternatively, the variation may be by addition or deletion of 1, 2, 3, 4, 5, 6, 7, 8, 9, 10, 11, 12, 13, 14, 15, 16, 17, 18, 19, 20 or more amino acids within the protein.

Amino acid substitutions may be conservative or non-conservative. Preferably, substitutions are conservative substitutions, in which one amino acid is substituted for another amino acid with similar structural and/or chemical properties. Exemplary conservative substitutions are listed below.

Ala (A) val; leu; ile
Arg (R) lys; gin; asn
Asn (N) gln; his; lys

### Asp (D) glu

### Cys (C) ser

### Gln (G) asn

### Glu (E) asp

Gly (G) pro; ala
His (H) asn; Gln; lys; arg
He (I) leu; val; met; ala
norleucine leu
Leu (L) norleucine; ile; met; ala; phe
Lys (K) arg; Gln; asn
Met (M) leu; phe; ile
Phe (F) leu; val; ile; ala; tyr

### Pro (P) ala

### Ser (S) thr

### Thr (T) ser

Trp (W) tyr; phe
Tyr (Y) trp; phe; thr; ser
Val (V) ile; leu; met; phe; ala; norleucine

Variant proteins may include proteins that have at least about 80% amino acid sequence identity with a polypeptide sequence disclosed herein. Preferably, a variant protein will have at least about 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length polypeptide sequence or a fragment of a polypeptide sequence as disclosed herein. Amino acid sequence identity is defined as the percentage of amino acid residues in the variant sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both. Methods for sequence alignment and determination of sequence identity are well known in the art, for example using publicly available computer software such as BioPerl, BLAST, BLAST-2, CS-BLAST, FASTA, ALIGN, ALIGN-2, LALIGN, Jaligner, matcher or Megalign (DNASTAR) software and alignment algorithms such as the Needleman-Wunsch and Smith-Waterman algorithms.

For example, the percentage identity may be calculated by performing a pairwise global alignment based on the Needleman-Wunsch alignment algorithm to find the optimum alignment (including gaps) of two sequences along their entire length, for instance using Needle, and using the BLOSUM62 matrix with a gap opening penalty of 10 and a gap extension penalty of 0.5.

Fragments of the proteins and variant proteins disclosed herein are also encompassed by the invention. Such fragments may be truncated at the N-terminus or C-terminus, or may lack internal residues, for example, when compared with a full length protein. Certain fragments lack amino acid residues that are not essential for enzymatic activity. Preferably, said fragments are at least about 1, 2, 3, 4, 5, 6, 7, 8, 9, 10, 20, 30, 40, 50, 60, 70, 80, 90, 100, 110, 120, 150, 250, 300, 350, 400, 450, 500 or more amino acids in length.

### Pharmaceutical Compositions and Administration

The peptide vaccines and antibodies of the invention may be formulated into a pharmaceutical form, preferably in combination with a pharmaceutically acceptable carrier. The pharmaceutical forms suitable for injectable use include sterile aqueous solutions or dispersions and sterile powders for the extemporaneous preparation of sterile injectable solutions or dispersions In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intraarterial, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, (see for example, Remington: The Science and Practice of Pharmacy, 21^{st} Edition, Lippincot and Williams, 2005). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, ‘carrier’ includes, without limitation, solvents, dispersion media, vehicles, coatings, diluents, antibacterial and antifungal agents, isotonic and absorption delaying agents, buffers, carrier solutions, suspensions, colloids, liposomes and virosomes such as those described in Bomsel et al (2011) Immunity 34: 269-280. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase ‘pharmaceutically-acceptable’ or ‘pharmacologically-acceptable’ refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

The vaccine composition will preferably contain an agent that enhances the protective efficacy of the vaccine, such as an adjuvant. Adjuvants include any compound or compounds that act to increase a protective immune response to the peptide antigen, thereby reducing the quantity of antigen necessary in the vaccine, and/or the frequency of administration necessary to generate a protective immune response. Adjuvants can include for example, emulsifiers; muramyl dipeptides; pyridine; aqueous adjuvants such as aluminum hydroxide; oxygen-containing metal salts; chitosan-based adjuvants, and any of the various saponins, oils, and other substances known in the art, such as Ampfaigen, LPS, bacterial cell wall extracts, bacterial DNA, CpG sequences, synthetic oligonucleotides and combinations thereof (Schijns et al (2000) Curr. Opin. Immunol, 12:456), Mycohacterialplilei (phlei) cell wall extract (CWE) (U.S. Pat. No. 4,744,984), M. phlei DNA (M-D A), and M-DNA-M phlei ceil wall complex (MCC), heat-labile enterotoxin (LT), cholera toxin (CT), cholera toxin B subunit (CTB). Compounds which can serve as emulsifiers include natural and synthetic emulsifying agents, as well as anionic, cationic and nonionic compounds. Oxygen-containing metal salts include salts of Al, K, Ca, Mg, Zn, Ba, Na, Li, B, Be, Fe, Si, Co, Cu, Ni, Ag, Au, and Cr which are sulphates, hydroxides, phosphates, nitrates, iodates, bromates, carbonates, hydrates, acetates, citrates, oxalates, and tartrates, and mixed forms thereof, including aluminium hydroxide, aluminium phosphate, aluminium sulphate, potassium aluminium sulphate, calcium phosphate, Maalox (mixture of aluminium hydroxide and magnesium hydroxide), beryllium hydroxide, zinc hydroxide, zinc carbonate, zinc chloride, and barium sulphate Among the synthetic compounds, anionic emulsifying agents include, for example, the potassium, sodium and ammonium sails of lauric and oleic acid, the calcium, magnesium and aluminum salts of fatty acids, and organic sulfonates such as sodium lauryl sulfate. Synthetic cationic agents include, for example, cetyltrhethylammonlum bromide, while synthetic nonionic agents are exemplified by glycerylesters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan. monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil can be a mineral oil, a vegetable oil, or an animal oil. Mineral oils are liquid hydrocarbons obtained from petrolatum via a distillation technique, and are also referred to in the art as liquid paraffin, liquid petrolatum, or white mineral oil. Suitable animal oils include, for example, cod liver oil, halibut oil, menhaden oil, orange roughy oil and shark liver oil, ail of which are available commercially. Suitable vegetable oils, include, for example, canola oil, almond oil, cottonseed oil, corn oil, olive oil, peanut oil, safflower oil, sesame oil, soybean oil, and the like. Freund's Complete Adjuvant (FCA) and Freund's incomplete Adjuvant (FIA) are two common adjuvants that are commonly used in vaccine preparations, and are also suitable for use in the present invention. Both FCA and FIA are water-in-mineral oil emulsions; however, FCA also contains a killed Mycobacterium sp. Particularly preferred adjuvants for mucosal vaccines include galactosyl ceramide (GalCer), as described in Lee et al (2011) Vaccine 29: 417-425.

Immunomodulatory cytokines can also be used in the vaccine compositions to enhance vaccine efficacy, for example, a an adjuvant, Non-limiting examples of such cytokines include interferon alpha (IFN-a), interleukin-2 (IL-2), and granulocyte macrophage-colony stimulating factor (GM-CSF), or combinations thereof. GM-CSF is preferred.

When provided prophylactically, the immunogenic compositions of the invention are ideally administered to a subject in advance of HIV infection, or evidence of HIV infection, or in advance of any symptom due to AIDS, especially in high-risk subjects. The prophylactic administration of the immunogenic compositions can serve to provide protective immunity of a subject against HIV-1 infection or to prevent or attenuate the progression of AIDS in a subject already infected with HIV-1. When provided therapeutically, the immunogenic compositions can serve to ameliorate and treat AIDS symptoms and are advantageously used as soon after infection as possible, preferably before appearance of any symptoms of AIDS but may also be used at (or after) the onset of the disease symptoms.

Administration may be via a parenteral or non-parenteral route. Routes of administration will vary, naturally, with the location and nature of the disease, and include, e.g. intravenous, intrarterial, intradermal, transdermal, intramuscular, mucosal subcutaneous, percutaneous, intratracheal, intraperitoneal, perfusion and lavage. Preferably, administration is via a mucosal route, for example via a nasal, oral (via the mucosa of the digestive system), vaginal, buccal, rectal, sublingual, ocular, urinal, pulmonal or otolar (vie the ear) route.

For nasal administration, an exemplary formulation may be a nasal spray, lavage, drop or squirt system such as the Bidose Liquid from Aptar, Pfeffer Group or the Accuspray from Becton Dickinson (see Brandztaeg, 2011, AJRCCM, Bitter et al (2011) Curr Probl Dermatol 40: 20-35).

The treatments may include various ‘unit doses.’ A unit dose is defined as containing a predetermined-quantity of the therapeutic composition comprising a lentiviral vector of the present invention. The quantity to be administered, and the particular route and formulation, are within the skill of those in the clinical arts. A unit dose need not be administered as a single injection but may comprise continuous infusion over a set period of time. A unit dose may contain at least 0.01, 0.05, 0.1, 0.5, 1.0, 2.5, 5.0, 10.0, 15.0, 25.0 or 50.0 mg of the active ingredient. Optionally, a unit dose contains less than 0.01, 0.05, 0.1, 0.5, 1.0, 2.5, 5.0, 10.0, 15.0, 25.0 or 50.0 mg of the active ingredient. In one embodiment, a unit dose contains from about 0.001 mg to about 50 mg of the active ingredient. In another embodiment a unit dose contains from about 1 mg to about 10 mg of active ingredient.

In one embodiment, the vaccine composition may be administered in a single daily dose, or the total daily dosage may be administered in divided doses, for example, two, three or four times daily. Furthermore, the vaccine composition may be administered in intranasal form via topical use of suitable intranasal vehicles, via transdermal routes, using those forms of transdermal skin patches well known to persons skilled in the art, by implantable pumps; or by any other suitable means of administration. To be administered in the form of a transdermal delivery system, for example, the dosage administration will, of course, be continuous rather than intermittent throughout the dosage regimen.

The dosage regimen utilizing the vaccine composition is selected in accordance with a variety of factors including type, species, age, weight, sex and medical condition of the patient; the severity of the condition to be treated; the route of administration; the renal and hepatic function of the patient; and the particular compound employed. A physician or veterinarian of ordinary skill can readily determine and prescribe the effective amount of the drug required to prevent, counter or arrest the progress of the disease or disorder that is being treated.

Vaccine administration may further comprise a prime-boost regimen. In these methods, one or more priming immunisations are followed by one or more boosting immunisations. The actual immunogenic composition can be the same or different for each immunisation and the type of immunogenic composition, the route, and formulation of the immunogens can also be varied. One useful prime-boost regimen provides for two priming immunisations, four weeks apart, followed by two boosting immunisations at 4 and 8 weeks after the last priming immunisation.

Such a regimen may comprise, for example, priming with full-length gp41 and boosting with one or more immunogenic peptides as disclosed herein. Either the prime or the bosst, or both, may be administered in the form of a DNA molecule encoding the peptide or polypeptide in question. Without being bound by theory, it is thought that this regimen might elicit gp41 cross-reactive antibodies targeted to the epitopes present in the priming immunogen. The designed protein fragments may be expressed in E. coli in order to prevent glycosylation and consequent epitope masking that might occur if expressed in a eukaryotic expression system. The use of E. coli to produce non-glycosylated versions of the invention may have contributed to the success of the approach.

Immunisation schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. In a particularly advantageous embodiment of the present invention, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks or 70 weeks.

The immunisation regimes typically have from 1 to 6 administrations of the immunogenic composition, but may have as few as 1, 2, 3, 4 or 5. The methods of inducing an immune response can also include administration of an adjuvant with the immunogens. In some instances, annual, biannual or other long interval (5-10 years) booster immunisation can supplement the initial immunisation protocol.

A specific embodiment of the invention provides methods of inducing an immune response against HIV in a subject by administering an immunogenic composition of the invention, preferably comprising one or more of the epitopes of the invention, one or more times to a subject wherein the epitopes are expressed at a level sufficient to induce a specific immune response in the subject. Such immunisations can be repeated multiple times at time intervals of at least 2, 4 or 6 weeks (or more) in accordance with a desired immunisation regime.

The immunogenic compositions of the invention can be administered alone, or can be co-administered, or sequentially administered, with other HIV immunogens and/or HIV immunogenic compositions, e.g., with ‘other’ immunological, antigenic or vaccine or therapeutic compositions thereby providing multivalent or ‘cocktail’ or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

When used in combination, the other HIV immunogens can be administered at the same time or at different times as part of an overall immunisation regime, e.g., as part of a prime-boost regimen or other immunisation protocol.

In one embodiment, the other HIV immunogen is env, preferably the HIV env trimer. Many other HIV immunogens are known in the art. One such immunogen is HIVA (described in WO 01/47955), which can be administered as a protein, on a plasmid (e.g., pTHr.HIVA) or in a viral vector (e.g., MVA.HIVA). Another such HIV immunogen is RENTA (described in PCT/US2004/037699), which can also be administered as a protein, on a plasmid (e.g., pTHr.RENTA) or in a viral vector (e.g., MVA.RENTA).

For example, one method of inducing an immune response against HIV in a human subject comprises administering at least one priming dose of an HIV immunogen and at least one boosting dose of an HIV immunogen, wherein the immunogen in each dose can be the same or different, provided that at least one of the immunogens is an epitope of the present invention, a nucleic acid encoding an epitope of the invention or an expression vector, preferably a VSV vector, encoding an epitope of the invention, and wherein the immunogens are administered in an amount or expressed at a level sufficient to induce an HIV-specific immune response in the subject. The HIV-specific immune response can include an HIV-specific T-cell immune response or an HIV-specific B-cell immune response. Such immunisations can be done at intervals, preferably of at least 2-6 or more weeks.

### Prevention and Treatment

‘Treatment’ includes both therapeutic treatment and prophylactic or preventative treatment, wherein the object is to prevent or slow down the targeted pathologic condition or disorder. Those in need of treatment include those already with the disorder as well as those prone to have the disorder or those in whom the disorder is to be prevented. The terms ‘therapy’, ‘therapeutic’, ‘treatment’ or ‘treating’ include reducing, alleviating or inhibiting or eliminating the symptoms or progress of a disease, as well as treatment intended to reduce, alleviate, inhibit or eliminate said symptoms or progress. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods and compositions of the invention are used to delay development of a disease or disorder or to slow the progression of a disease or disorder.

Treatment in accordance with the invention includes a method of treating a cancer or other neoplastic disorder which comprises administering to a patient in need of treatment a peptide, nucleic acid, antibody composition of the invention. Preferably, the treatment further comprises administering to said patient a chemotherapeutic drug, preferably a drug in prodrug form. The two components may be administered together, for example in the form of a combined pill, or separately. Administration may be sequential or simultaneous. ‘Sequential’ administration indicates that the components are administered at different times or time points, which may nonetheless be overlapping. Simultaneous administration indicates that the components are administered at the same time.

Preferably, an effective amount, preferably a therapeutically effective amount of the protein or vector of the invention is administered. An ‘effective amount’ refers to an amount effective, at dosages and for periods of time necessary, to achieve the desired therapeutic or prophylactic result. The effective amount may vary according to the drug or prodrug with which the protein or vector is co-administered.

A ‘therapeutically effective amount’ of a protein or vector of the invention may vary according to factors such as the disease state, age, sex, and weight of the individual, and the ability of the protein, to elicit a desired therapeutic result. A therapeutically effective amount encompasses an amount in which any toxic or detrimental effects of the protein are outweighed by the therapeutically beneficial effects. A therapeutically effective amount also encompasses an amount sufficient to confer benefit, e.g., clinical benefit.

Throughout the specification, terms such as ‘comprises’, ‘comprised’, ‘comprising’ and can have the meaning attributed to them in most patent jurisdictions, preferably in the jurisdiction in question; e.g. they can mean ‘includes’, ‘included’, ‘including’, etc. Terms such as ‘consisting of’ ‘consisting essentially of’ and ‘consists essentially of have the meaning ascribed to them in most patent jurisdictions, preferably in the jurisdiction in question; e.g., they may imply the exclusion of all, most, or all but a negligible amount of other elements, or they may allow for elements not explicitly recited, but exclude elements that are found in the prior art or that affect a basic or novel characteristic of the invention.

An ‘isolated’ peptide, protein or nucleic acid may be isolated substantially or completely away from one or more elements with which is associated in nature, such as other naturally occurring peptide, protein or nucleic acids or other peptide or nucleic acid sequences.

The term ‘about’ as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The invention will now be described in more detail by means of the following non-limiting figures and examples. All patent and literature references cited herein are hereby incorporated by reference in their entirety.

### BRIEF DESCRIPTION OF THE FIGURES

FIGS. 1-4: 2F5 IgA2 binds to gp41MPER more robustly than 2F5 IgG1.

FIG. 1: 2F5 IgG1 and 2F5 IgA2 binding to P1 and ELDKWA occurs in a dose dependent manner. The specificity of 2F5 IgG1 (light grey) and of 2F5 IgA2 (black) for P1 (solid square, solid line) and for ELDKWA (solid square, broken line) was evaluated by ELISA. W₆₆₆A-mutated P1 (solid square, dotted line) was used as negative control. Goat anti-human IgG or IgA were used as detection reagent to allow for direct comparison of both 2F5 isotypes. Specific binding (OD450 nm) is plotted as a function of 2F5 Ab concentration (nM). One representative experiment of at least three performed in duplicate is shown.

FIG. 2: Competitive binding of 2F5 IgA2 to P1 and ELDKWA in the presence of an excess of 2F5 IgG1 (solid square, solid line) or irrelevant IgG (solid square, dotted line) used as negative control, was evaluated in competitive ELISA. 2F5 IgA2 specific binding (OD450 nm) is plotted as function of the competitor IgG concentration (nM). One representative experiment of at least three performed in duplicate is shown.

FIG. 3: Competitive binding of 2F5 IgA2 to P1 gp41 in the presence of an excess of 2F5 IgG1 (solid square, solid line) or irrelevant IgG (solid square, dotted line) used as negative control, was evaluated in competitive ELISA. 2F5 IgA2 specific binding (OD450 nm) is plotted as function of the competitor IgG concentration (nM). One representative experiment of at least three performed in duplicate is shown.

FIG. 4: 2F5 IgA2 and IgG1 specific binding to HIV-1 JR-CSF virions. 2F5 IgG1 (grey bars) or IgA2 (black bars) bound to goat anti-human IgG or IgA coated ELISA plates, were incubated with HIV-1 JR-CSF for 1 hr at 37° C. HIV-1 binding was evaluated by measuring the p24 content after removal of unbound virus. Results are presented as HIV-1 p24 captured by 2F5 IgA and IgG1, resp.; after subtraction of the nonspecific binding (HIV-1 p24 captured by irrelevant IgA and IgG, resp.). Values represent the mean±SD of two experiments performed in triplicate.

FIGS. 5-8: 2F5 IgA2 and IgG1 have different anti-viral efficiencies.

FIG. 5: Transcytosis of HIV-1 induced by HIV-1 R5-infected PBMCs across epithelial cells for 2 hrs was measured after preincubation of HIV+PBMCs with 2F5 IgG1 (grey bars) or IgA2 (black bars). Results are presented as % of transcytosis blockade in the absence of Abs. *p=0.006, **p=0.005, ***p=0.004. Values represent the mean±SD of three independent experiments performed in duplicate.

FIG. 6: Inhibition of HIV-1 transfer from Langerhans cells (LCs) to autologous CD4+ T cells evaluated by measuring the p24 released by LCs/T cells co-cultures at day 5. HIV-1 JR-CSF was pre-incubated with LCs for 2 hr before addition of the antibodies, either alone (2F5 IgG1—grey bars, 2F5 IgA2—black bars) or in combination (hatched bars), and T cells. The % of neutralization was defined as the % of transfer inhibition in the absence of Abs. Values represent the mean±SD of three independent experiments performed in duplicate.

FIG. 7: Dose dependent inhibition of HIV-1 neutralization mediated by 2F5-Abs assessed in a single cycle infectivity assay using p24 staining on primary CD4+ T cells. 2F5 IgG1 (grey bars) IgA2 (black bars) or their combination (hatched bars) were incubated for 1 hr at 37° C. with HIV-1JR-CSF before addition of CD4+ cells for 36 hr. The % of neutralization was defined as the reduction of p24⁺ cells compared with control-infected cells in the absence of Abs. Values represent the mean±SD of three independent experiments performed in triplicate.

FIG. 8: Dose dependent inhibition of HIV-1 neutralization mediated by 2F5-Abs assessed in a single cycle infectivity assay using p24 staining on CEM-CCR5+ cell line. 2F5 IgG1 (grey bars) IgA2 (black bars) or their combination (hatched bars), were incubated for 1 hr at 37° C. with HIV-1JR-CSF before addition of CD4+ cells for 36 hr. The % of neutralization was defined as the reduction of p24⁺ cells compared with control-infected cells in the absence of Abs. *p=0.0023, **p=0.001, ***p=0.00045. Values represent the mean±SD of three independent experiments performed in triplicate.

FIGS. 9-10: Identification of epitopes bound by 2F5 IgA2 and 2F5 IgG1

FIG. 9: Multiple alignment of sequences specific for gp 41 and IgA2. Sequences of epitopes retrieved after panning the random 12-mer library on 2F5 IgA2 that were used for in silico analyses. The multiple alignment on selected sequences was performed with the t_coffee server [37] using the gp41 C-helix as reference. ‘Frequency’ refers to the retrieval occurrence obtained for each sequence, respectively.

FIG. 10. Multiple alignment of sequences specific for gp 41 and 2F5 IgG1. Sequences of epitopes retrieved after panning the random 12-mer library on 2F5 IgG1 that were used for in silico analyses. ‘Frequency’ refers to the retrieval occurrence obtained for each sequence, respectively.

### BRIEF DESCRIPTION OF THE SEQUENCE LISTING

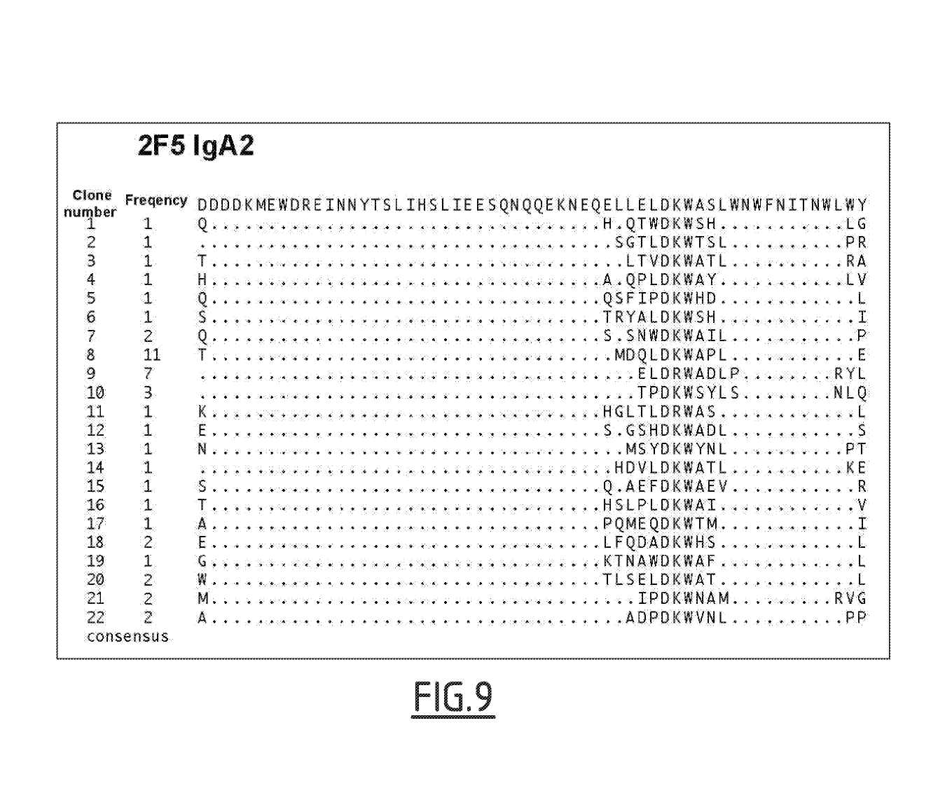

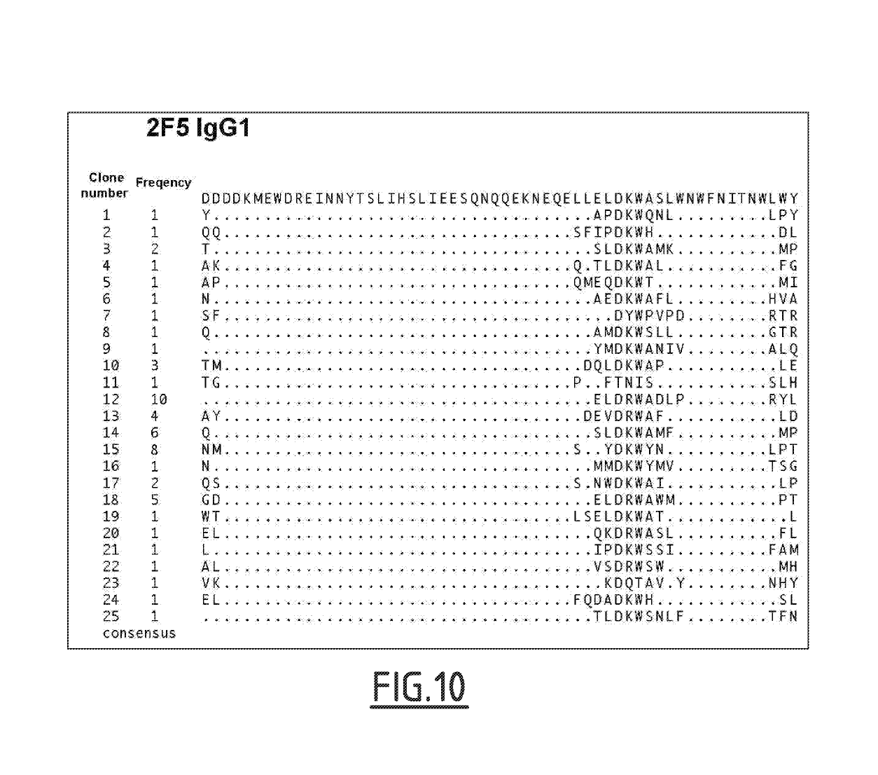

SEQ ID Nos 1-22: epitope sequences 1-22 disclosed in FIG. 9 (2F5 IgA2)
SEQ ID Nos 23-47: epitope sequences 1-25 disclosed in FIG. 10 (2F5 IgG1)
SEQ ID No 48 gp41 helix 1
SEQ ID No 49 gp41 helix 2

### SEQ ID No 50 2F5a VH CDR1

### SEQ ID No 51 2F5a VH CDR2

### SEQ ID No 52 2F5a VH CDR3

### SEQ ID No 53 2F5a VL CDR1

### SEQ ID No 54 2F5a VL CDR2

### SEQ ID No 55 2F5a VL CDR3

### SEQ ID No 56 2F5a VH

### SEQ ID No 57 2F5a VL

SEQ ID No 58-63: peptides described in the Examples.

### EXPERIMENTAL

### Materials and Methods

### Peptides and Recombinant Proteins

Peptide ELDKWA, P1 (aa 630-685) Glade B HXB2 HIV-1 and W₆₆₆A mutated P1 (Alfsen and Bomsel 2002, J Biol Chem 277: 25649-59) were chemically synthesised (purity >95%) by Eurogentec (Belgium). Alternatively, P1 was derived at its C terminus by addition of a cysteine, allowing for coupling with 1,3-dipalmitoyl-glycero-2-phosphoethanolamine (synthesis purity of >92%, Bachem, Bubendorf, Switzerland). This derived P1 is hence referred to as P1-PE (Coutant, Yu et al. 2008).

The trimeric recombinant gp41 construct was kindly provided by Mymetics Corp and Protein X′ pert (Grenoble, France).

### Cloning of 2F5 Antibody V Regions, Expression of 2F5 IgG1 and IgA2 Antibodies and Sequence Analysis

DNA of 2F5 antibody V regions, VJ (for the light chain) and VDJ (for the heavy chain), were synthesized as previously described (Kunert, Ruker et al. 1998 AIDS Res Hum Retroviruses 14(13): 1115-28) and codon-optimized for expression in eukaryotic cells (Pasteur-Mérieux Conaught, France). For the production of 2F5 IgG1, DNA was cloned into two vectors, VHExpress and VKExpress, which direct the synthesis of the human gamma-1 heavy chain and light chain respectively.

For the 2F5 IgA2, DNA of 2F5 V regions was next cloned into the pcDNA3:VHCα2m(1) and pcDNA3:VLCα (Berdoz, Blanc et al. 1999, Proc Natl Acad Sci USA 96(6): 3029-34) provided by B. Corthésy (Swiss Institute for Experimental Cancer Research, Lausanne, Switzerland).

Monomeric 2F5 IgA2 and IgG1 were expressed in CHO dhfr− cells (CHO) cultured in RPMI medium supplemented with 10% fetal calf serum and in the presence of G418 mycophenolic acid as selection antibiotics.

Sequencing of 2F5 antibodies was performed on purified plasmid DNA with an automated DNA sequencer using appropriate primers (Applied Biosystems).

### ELISA

The concentration of 2F5 antibodies was measured by sandwich ELISA using goat anti-human IgA (Caltag, France) or IgG (Jackson ImmunoResearch Laboratories Inc. UK) and biotinylated mouse anti-human Ig kappa light chains (B.D. Pharmingen, USA) as described (Tudor, Derrien et al. 2009, Mucosal Immunol 2(5): 412-26).

Specificity of the antibodies was measured by coating microtiter plates (NUNC-Immuno Plate MaxiSorp Surface, or Peptide Immobilizer Exiqon (Denmark)) with either recombinant gp41 (trimeric rgp41 at 0.25 microg/well), peptide P1 (0.1 microg/well), peptide ELDKWA (SEQ ID No 58) (0.05 microg/well) overnight at 4° C. in carbonate buffer pH9. Antibody binding was detected with biotinylated-mouse anti-human IgA or IgG specific heavy chains secondary antibody (Jackson ImmunoResearch Laboratories Inc. UK) followed by streptavidin-HRP (horseradish peroxidase).

For competitive binding ELISA, 2F5-IgG1, at indicated concentrations, were used to compete with a constant concentration of 2F5-IgA2 (0.027 nM) for the binding to P1, ELDKWA or to rgp41, respectively. Binding of competing Ab was detected with a byotinilated mouse anti-human IgA followed by streptavidin-HRP.

Importantly, the 2F5 IgG1 had similar binding activities to both ELDKWA and P1 to the reference 2F5 IgG1 (Purtscher, Trkola et al. 1994, AIDS Res Hum Retroviruses 10(12): 1651-8.)

The affinity of 2F5 IgG1 and 2F5 IgA2 for P1-PE inserted in liposomes was evaluated as described earlier (Coutant, Yu et al. 2008, Faseb J 22(12): 4338-51). Briefly, immobilizer amino plates (Nunc, Rochester, N.Y., USA) were coated with streptavidin overnight at 4° C. After blocking, liposomes with or without P1-PE were allowed to attach to streptavidin-coated plates by incubation overnight at 4° C. A similar quantity of P1-PE or alternatively biotinylated P1, was added per well for all conditions of the P1-PE/lipid ratio. Specific binding was detected with HRP conjugated goat anti-human IgG or IgA (Jackson ImmunoResearch, West Grove, Pa., USA) and by reading the absorbance at 490 nm using a plate reader SpectraMax 340PC (Molecular Devices, Sunnyvale, Calif., USA).

Kd was obtained using GraphPad Prism 5.0 (GraphPad, San Diego, Calif., USA) as described (Coutant, Yu et al. 2008), by nonlinear regression of the data obtained by ELISA, after subtraction of nonspecific binding of 2F5-IgG1 and 2F5-Ig-A to liposomes devoid of P1-PE. Such computing is based on an equal amount of peptide bound on the surface of each well of the ELISA.

Virus capture was adapted from (Cavacini, Peterson et al. 1999). To capture 2F5Abs, ELISA plates (NUNC-Immuno Plate MaxiSorp Surface, Denmark) were coated overnight with goat anti-human IgG (H+L) or goat anti-human serum IgA alpha chain specific (Jackson ImmunoResearch Lab., PA, USA). After blocking, 2F5 Abs IgG1 and IgA2 at 1 microg/ml, were incubated for 1 hr at RT. Normal serum IgG or purified IgA2 were used as negative controls. HIV-1JR-CSF (clade B, R5 tropic) at 300 ng or 30 ng p24 was then added for 1 hr at 37° C. After washing of unbound virus, p24 was released from the captured virus by incubation with NP40 diluted in RPMI 1640 containing 10% fetal bovine serum. Released p24 was measured using a commercial ELISA (Innotest HIV-1 Antigen mAb, Innogentetics) according to manufacturer instruction.

### Target Cells

CD4+ T cells were purified from peripheral blood samples from 20 healthy donors using human CD4+ T cells enrichment kits (StemCell Technologies Inc., France) and stimulated for 2 days with 5 microg/ml phytohemagglutinin (Sigma-Aldrich, St. Louis, Mo.).

CEM-CCR5+ cells (AIDS Research and Reference Program, NIH) were maintained in RPMI 1640 containing 10% fetal bovine serum on a precise passage regimen (1:10 split, twice a week). To prepare cells for neutralization assays, cells were split 1:3 on the day of passage and used the following day.

### Virus Stock Preparation

A stock of HIV-1JR-CSF (clade B, R5 tropic) was prepared by transfecting 293T cells with a plasmid containing the DNA sequence of JR-CSF (NIH, Germantown, Md. USA) (Tudor, Derrien et al. 2009). Virus concentration was quantified by measuring p24 antigen using a commercial ELISA (Innotest HIV-1 Antigen mAb, Innogentetics) according to manufacturer instruction. The HIV-1 primary isolate 93BR029 (clade B, R5), obtained through the NIH AIDS Reagent Program, was amplified on PBMCs, as previously described (Magerus-Chatinet, Yu et al. 2007).

### HIV-1 Neutralization Assays

HIV-1 transcytosis: Inhibition of HIV-1 transcytosis across monolayers of the HEC-1 endometrial epithelial cell line induced upon contact with HIV-1-infected PBMC with appropriated controls, was performed as previously described (Bomsel, Pastori et al. 2007, Aids 21(1): 13-22) (Alfsen, Iniguez et al. 2001, J Immunol 166(10): 6257-65.).

Inhibition of HIV-1 Transfer from Langerhans Cells to T Cells.

The inhibition of HIV-1 transfer from Langerhans cells to T cells was evaluated using monocytes derived Langerhans cells (LCs) obtained from PBMCs as previously described (Geissmann, Prost et al. 1998) and autologous CD4+ T cells obtained by negative selection using human CD4+ T cells enrichment kit (StemCell Technologies Inc., France). Briefly, LCs were incubated with HIV-1 JR-CSF for 2 hr at 37° C., washed extensively to remove the free virus and distributed in 96 well plates at 100,000 cells/well. 2F5 IgG1 and IgA2 antibodies of the desired concentration, or mixtures containing equal proportions of both isotypes to achieve the concentration of a single antibody, were then added to the corresponding wells. Finally, either medium alone or resting CD4+ T cells in medium (100,000 cells/well) was added. The LCs-T co-cultures were incubated at 37° C. for 7 days. The virus transfer was evaluated by measuring p24-Ag using a commercial ELISA (Innotest HIV-1 Antigen mAb, Innogentetics) according to manufacturer instruction. Results are expressed as % of transfer in absence of Abs.

Single-Cycle Neutralization Assay:

The neutralization activity of 2F5 IgG1 and IgA2 was evaluated on purified CD4+ T cells and on CEM-CCR5+ infected with HIV-1 JR-CSF, by flow cytometric detection of intracellular p24-Ag as described (Tudor, Derrien et al. 2009).

For both target cells, at least four independent experiments, each point performed in triplicate, were performed.

Neutralization was defined in % of cells infected in absence of antibody. The neutralizing titer was defined as the respective inhibitory concentration (IC) 50, 90, and 99 of the antibody (interpolated between successive dilutions performed in triplicate) that neutralized infection by 50, 90 and 99%.

Neutralization Assays in TZM-bl Cells with Envelope-Pseudotyped Viruses:

Neutralization was also evaluated using a very sensitive test based on viruses pseudo typed with an HIV-1 envelope, here the HIV-1 R5 tropic QH0692.42 envelope. Neutralization correlates directly to the reduction in luciferase reporter gene expression after a single round of virus infection in JC53-BL cells (also termed TZM-bl cells) as described previously (Bomsel, Pastori et al. 2007). The antibody concentrations causing 50% (IC50), and 90% (IC90) reductions in luciferase reporter gene production were determined by regression analysis.

### Epitope Mapping

Epitope mapping of both isotypes was performed as described by (Humbert, Rasmussen et al. 2008), using a linear 12-mer peptide libraries displayed on the protein pill of M13 phages (New England Biolabs, Ipswich Mass., USA) and as recommended by the manufacturer. Briefly, IgA or IgG coated beads were incubated with 2F5 IgA2 or IgG1, 2 microg for the first round, 1 microg for the second and 0.5 microg for third rounds, in PBS/BSA 0.1%/Tween-20, 0.05% on a rotating wheel for 2 hrs at room temperature. After washing with PBS/BSA 0.1%/Tween-20, 0.05% epitope screening was initiated by incubating each of the beads 2F5 IgA2 or IgG1 with 10 microl of the original 12-mer (1×10¹¹) phage-displayed peptide library containing different phages, on a rotating wheel overnight at 4° C. Beads were then washed and bound phages were eluted by pH shift with 0.2 M glycine-HCl pH 2.2, and immediately neutralized with 1M Tris-HCl pH 9.1 (Sigma-Aldrich). Eluted phages were subjected to negative selection using beads coated with normal human IgA or IgG (Jackson ImmunoResearch Laboratories Inc. UK). Phages remaining from the negative selection were amplified in Escherichia coli (Escherichia coli ER2738, New England Biolabs), precipitated overnight at 4° C. with 20% PEG-8000/2.5 M NaCl (Sigma) and used for a second and third round of selection similar to the first, but with increased buffer stringency (Tween-20 concentration raised to 0.1 and 0.5% for the second and third round, resp.). After the third positive selection, the phages were titered, and single clones were picked and tested by phage ELISA for specific binding on each 2F5 IgA2 or IgG1, resp. Positives clones were amplified, and each specific peptide insert sequenced.

Mimotope Sequence Analyses and Mapping on gp41

For each set of 2F5 IgA2 and IgG1 mimotopes, epitope mapping on HIV-1 gp41 was performed using the pepitope software (http://pepitope.tau.acil) (Mayrose, Penn et al. 2007, Bioinformatics 23(23): 3244-6) and the recently published structure of a trimeric gp41 (Buzon, Natrajan et al. 2010, PLoS Pathog 6(5): e1000880) (PDB code: 2×7R). The Pepitope software is a web-based tool that aims at predicting discontinuous epitopes based on a set of peptides that were affinity selected against monoclonal antibody of interest. The software proposes two algorithms (PepSurf and Mapitope) to predict discontinuous epitopes, which rationale is that the set of peptides mimics the genuine epitope in terms of physicochemical properties and spatial organization. PepSurf algorithm (Mayrose, Shlomi et al. 2007) maps a peptide to the antigen surface by a systematic search of tridimensional compatible pathways. Resulting alignments are then scored and clustered to locate the epitope. In the Mapitope algorithm (Bublil, Freund et al. 2007), epitope key residues are determined by considering the whole candidate mimotope set. First, each mimotope is deconvolved into amino-acids pairs (AAP). Then, Mapitope identifies significantly over-represented AAP and map it onto the antigen surface to identify the epitope. Analyses were performed taking into account the screening occurrence for each peptide. Studies were carried out with both algorithms, but only PepSurf results are shown as PepSurf gave more robust and sensitive data. The pepitope program takes into account the number of times each sequence peptide was isolated during the experiment and provides 0 to N clusters that reveals key residues location. All predicted clusters were considered and amino acid predicted frequencies calculated (amino acid predicted occurrences normalized by the highest occurrence) for each chain of the gp41 structure. These frequencies were plotted all along the resolved monomer structure sequence and mapped onto the protein structure to visualize the epitope in the surface of gp41.

### Statistics

P values were calculated using the Student-t test.

### In Vivo Testing of Mimotope Immunogenicity

11 of the 2F5IgA2 mimotopes and 8 of the 2F5IgG1 mimotopes were tested in mice for immunogenicity in a prime-boost regimen with recombinant gp41.

The 11 2F5IgA2 mimotopes tested corresponded to clone numbers 2, 7, 8, 9, 10, 14, 17, 18, 20, 21 and 22 of FIG. 9 (SEQ ID NOs 2, 7, 8, 9, 10, 14, 17, 18, 20, 21 and 22)

The 8 2F5IgG1 mimotopes corresponded to clone numbers 3, 10, 12, 13, 14, 15, 17 and 18 of FIG. 10 (SEQ ID NOs 25, 32, 34, 35, 36, 37, 39 and 40).

Mice (BALB/cAnNCrl Female 8 w.o., Charles River) were immunized using conventional techiques (Li, BMC Biotechnology 2010, 10:84-90; Larralde, J. Virological Methods (2007) 140: 49-58; van Houten, Vaccine (2006) 24: 4188-4200; Galfré, Method in Enzymol, 1996 267:109-15) via the IM route for the priming with 10 microg of a recombinant gp41 protein (Ananza) followed by 5 successive boosts, 2 weeks apart with M13-phages expressing epitopes (10̂12 phages with equivalent quantity of each single mimotope/100 ul/injection) with or without adjuvant (pCCL28, 50 ug DNA/injection). Serum was collected a day 75 and analyzed by standard techniques in ELISA against a recombinant gp41 protein (Tudor et al, PNAS, 2012).

### Results

### Construction of 2F5 IgG1 and IgA2 Antibodies

Using the integrated vector system made up of VH Express and VK Express vectors under the control of EF-1αpromoter (Persic, Roberts et al. 1997, Gene 187(1): 9-18), and pcDNA3:VHCα2m(1) and pcDNA3:VLC α (Berdoz, Blanc et al. 1999, Proc Natl Acad Sci USA 96(6): 3029-34), the inventors successfully cloned 2F5 IgG1 and IgA2 isotypes, respectively. Functional monomeric 2F5-IgG1 and 2F5 IgA2 antibodies were produced in mammalian CHO cells. The presence of complete, covalently linked antibodies in the supernatants was confirmed by sandwich ELISA (not shown).

2F5 IgA2 had Higher Affinity than 2F5 IgG1 for the gp41MPER

Binding of both 2F5 IgG1 and IgA2 to (i) ELDKWA (SEQ ID No 58), the nominal 2F5 IgG epitope (Purtscher, Trkola et al. 1996, Aids 10(6): 587-93), (ii) P1, the minimal MPER that allows for HIV-1 binding to galactosyl ceramide, the HIV-1 mucosal receptor (Alfsen, Iniguez et al. 2001, J Immunol 166(10): 6257-65), and (iii) a trimeric recombinant gp41 protein (Tudor, Derrien et al. 2009) were evaluated comparatively in ELISA. W₆₆₆A mutated P1 (Alfsen, Iniguez et al. 2001, J Immunol 166(10): 6257-65) was used as negative control.

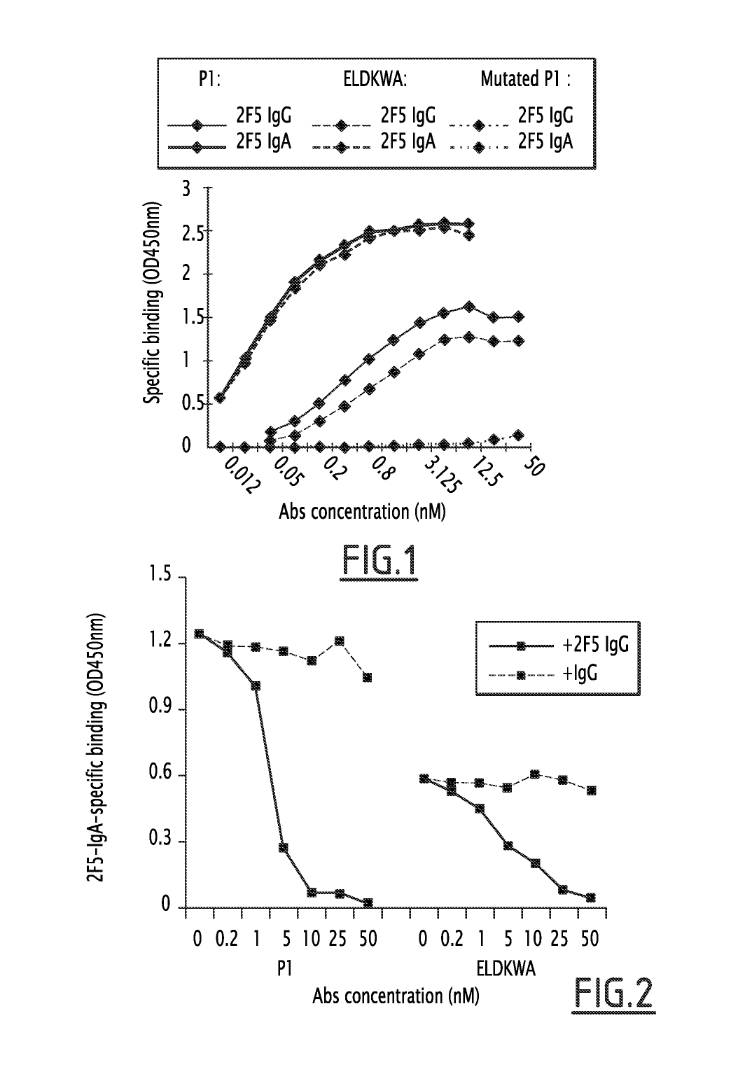

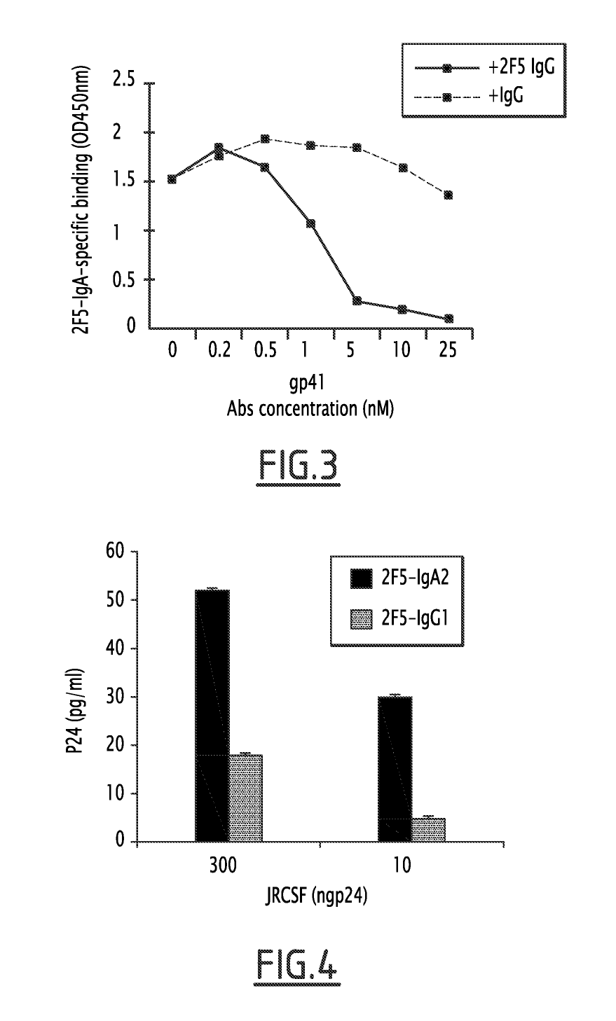

Both 2F5 IgG1 and IgA2 bound specifically to ELDKWA and P1 in a dose dependent manner (FIG. 1), but with a >100 fold higher efficacy for the IgA2 as compared to IgG1. Thus, 2F5 IgA2 binding activity at 0.05 nM was equivalent to that of its IgG1 counterpart at 6.25 nM (FIG. 1). Since both isotypes have identical V regions, these results suggested that other regions of the antibody could participate in antigen binding. Therefore, 2F5 IgA2 binding to ELDKWA, P1 and the trimeric rgp41 were evaluated by competition ELISA in the presence of an excess of 2F5 IgG1. An irrelevant human IgG was used as control. Twenty-five, 10 and 5 nM of 2F5 IgG1 were needed to compete with 0.027 nM of 2F5IgA2 binding to ELDKWA, P1 (FIG. 2) and rgp41 (FIG. 3), respectively, while no competition was observed with an irrelevant human IgG (FIG. 2, 3). Noteworthy, the longer the antigen, and therefore the more structured, the lower the IgG1 competing concentration was required to displace IgA2 binding.

At the virus or infected cell surface, the HIV envelope spike is organized as an oligomer, inserted in the viral or cell membrane bilayer with a specific lipid environment. Accordingly, the structure of P1 is lipid dependent. To mimic the bilayer anchor of P1, it was chemically coupled to phosphatidyl ethanolamine (PE) via its C-terminal end. The resulting P1-PE was inserted into liposomes of various composition mimicking either the viral membrane rich in cholesterol, di-hydro-sphingomyelin (DHSM), plasmalogen (pl)-PE and phosphatidyl serine (PS) (referred to as viral liposomes), or target cell membranes: either epithelial cells rich in glycosphingolipid (raft liposomes), or CD4+ T lymphocytes rich in phospholipids (nonraft liposomes) (see Table I). The PE anchor allows for free lateral movement and oligomerization of P1 at the liposome surface. The inventors thus evaluated the affinities of 2F5 IgG1 and IgA2 to the various P1-containing liposomes in comparison to P1 or liposomes without the P1-PE antigen.

The 2F5 IgA2 affinity for P1 alone was more than 10 times higher compared to 2F5 IgG1 (with respective Kd=0.2 and 7.3 nM, Table 2). For both antibodies, affinities to P1-PE inserted into the various liposomes were one order of magnitude higher than that of binding to P1 in solution (Table 2). However, the difference in affinities between the two isotype remains identical in the presence of lipids mimicking various membrane contexts, 10 times higher for the IgA2 as compared to IgG1 (Table 2). Importantly, no binding of 2F5 IgG1 or IgA2 was detectable on liposome lacking P1-PE (not shown), suggesting that antibodies are not poly specific.

These results highlight the importance of the lipid context in 2F5 antibodies affinities, and the greater affinity of 2F5 IgA2 for P1 as compared to the IgG1 isotype, irrespective of the lipid context. As 2F5 IgG1 and IgA2 have identical V region, the ELISA results suggest that the constant region (CH1) of the 2F5 antibody participates to antigen binding.

2F5 IgA2 Had Higher Affinity than 2F5 IgG1 for the Free Virus

Considering the importance of the lipid context in 2F5 antibodies affinities and trying to be closer to the in vivo condition, the next step was to assess the affinity of 2F5 isotypes for the free virus. Using virus capture ELISA, our results showed that both 2F5 IgG1 and IgA2 isotypes bound specifically to R5 tropic HIV-1 JR-CSF, and, again, in this ELISA system IgA2 exhibits a higher affinity than IgG1 (FIG. 4).

2F5 IgA2 and IgG1 have Different Antiviral Efficiencies

Antiviral activities are not restricted to neutralization of infection, and may involve either the variable region of the antibody—as it occurs in blockade of HIV-1 transcytosis, or in inhibition of virus transfer from cell to cell via a viral synapse—or in addition, its constant region as in antibody dependent cell cytotoxicity (ADCC). Therefore, the various antiviral activities were evaluated comparatively for the 2F5 IgG1 and IgA2 isotypes.

2F5-IgA2 Blocks HIV-1 Transcytosis More Efficiently than 2F5-IgG1

HIV translocation across mucosal barrier depends on the structure of the covering epithelium. Monostratified epithelial cells translocate the virus from the apical to the basolateral surface via vesicular transcytosis without epithelial cell infection. Transcytosis blockade by 2F5 IgA2 and IgG1 of cell free HIV-1 or initiated upon contact with HIV-1 infected PBMCs was evaluated, comparatively.

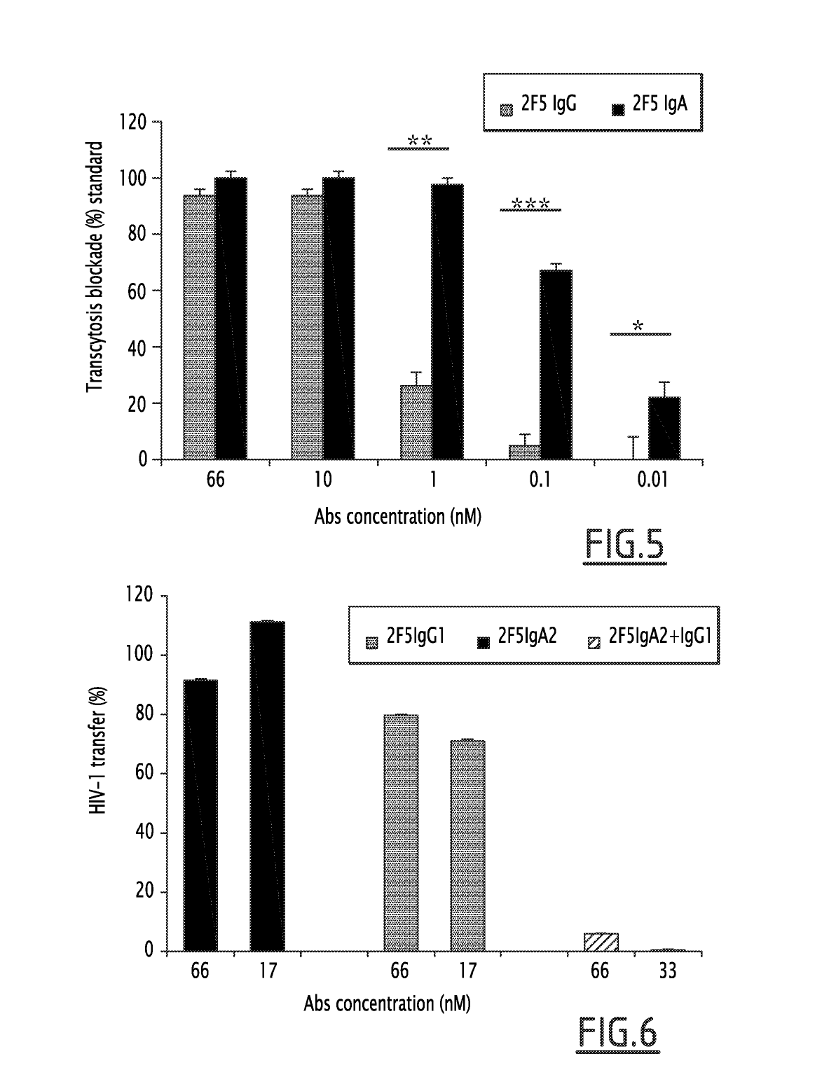

HIV-1 clade B R5 infected PBMCs were pre-incubated with each one of 2F5 isotype, and added to the apical pole of HEC-1 endometrial epithelial cells grown as a tight polarized epithelial barrier on permeable support. After 2 hrs, virus transcytosis was quantified by measuring p24-Ag in the basolateral chamber (Alfsen, Iniguez et al. 2001, J Immunol 166(10): 6257-65), (Bomsel, Tudor et al. 2011, Immunity 34(2): 269-80). Both 2F5 antibodies blocked virus transcytosis, but in a more robust manner for the IgA2 as compared to the IgG1 (IC90=1 nM and 10 nM resp.) (FIG. 5). Additionally, cell-free HIV transcytosis, although 100 times less efficient than when induced from HIV-1 infected cells, was also better blocked by 2F5 IgA2 as compared to IgG1 (not shown).

2F5-IgG1 and IgA2 Act Synergistically to Block the HIV-1 Transfer from Langerhans Cells to T Cells

Intraepithelial CD4+ T cells and LCs are primary targets of HIV-1 infection in the human vaginal epithelium. Intraepithelial CD4+ T cells can bind and take up the CCR5-tropic HIV-1 either independent of LCs, or following infectious synapse formation between the two cells types, suggesting that LCs can spread infection to T cells.

The inventors therefore evaluated whether 2F5 IgG1 and IgA2 could block virus transfer from LCs to CD4+ T cells in vitro. Langerhans cells where first incubated with CCR5-tropic HIV-1 clade B (JR-CSF) for 2 hr. After washing to eliminate the free virus, different concentrations of 2F5-IgA2 and IgG1 were added, either alone or in combination, and LCs were further cultured with autologous CD4+ T lymphocytes for 7 days at 37° C.

2F5 as IgG1 or IgA2 at 17 or 66 nM were not able to efficiently block HIV-1 transfer to autologous CD4+ T lymphocytes (although a limited (30%) block was found for IgG1 at 66 nM) (FIG. 6). In contrast, combining both isotypes in equal proportion to reach 17 nM or 66 nM, completely inhibited the virus transfer (FIG. 6).

These results demonstrate that the 2F5 IgG1 and IgA2 act synergistically to inhibit HIV-1 transfer from LCs to CD4+ T lymphocytes.

2F5 IgA2 Neutralizes CD4+ T Cell Infection by R5 JR-CSF HIV-1 Better than 2F5-IgG1

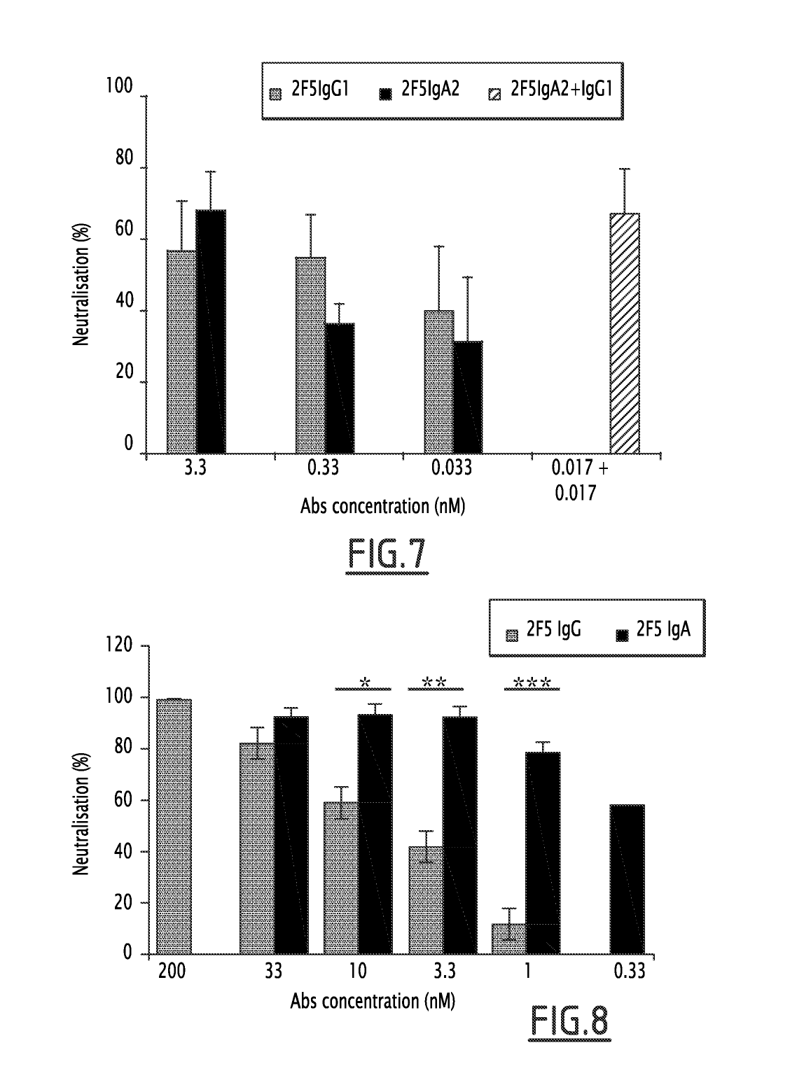

Considering the pivotal role of the CD4 cells in immune response regulation, their earliest depletion at mucosal level could compromise the mucosal defense, including the barrier function of epithelial cells. The potential of 2F5 IgA2 and IgG1 to neutralize CD4+ T cell HIV-1 infection was evaluated on primary CD4+ T cells using low concentrations of both isotypes. 2F5 IgA2 and IgG1 neutralized the infection of CD4+ T cells in a dose dependent manner (FIG. 7). Here again, a robust neutralization was obtained at low concentrations combination of both isotypes to reach 0.033 nM, suggesting a synergistic effect (FIG. 7).

The neutralization activity of both 2F5 antibody isotypes was also evaluated on CEM-CCR5+ cells. Earlier studies demonstrated that the CEM-CCR5+ lymphocytic cells expressing CD4, and both HIV-1 co-receptors, CXCR4 and CCR5, were a preferable model to evaluate HIV infection and neutralization over activated PBMCs or purified CD4+ T cells, due to elimination of donor-to-donor variation in HIV-1 replication efficiency. CEM-CCR5+ cells efficiently replicate HIV-1 JR-CSF strain, with around 8% infected cells after infection with 75 ng of HIV-1 p24 for 36 hr in a neutralization assay based on a single cycle infectivity test (Holl, Hemmerter et al. 2004, J Immunol 173(10): 6274-83) (Tudor, Derrien et al. 2009, Mucosal Immunol 2(5): 412-26). Furthermore, using CEM CCR5+ cell line allows for more reproducible and precise evaluation of neutralization in a single-round infection assay. As control, preincubation of the virus with AZT completely inhibited infection to a level comparable to that of mock-infected cells (not shown).

Compared to primary CD4+ T cells (FIG. 7), higher antibodies concentrations, starting with 200 nM for 2F5 IgG1 and 33 nM for 2F5 IgA2, were used. Both 2F5 antibody isotypes neutralized infection of CEM-CCR5+ target cells in a dose dependent manner (FIG. 8). Importantly, 2F5 IgA2 neutralized infection more robustly than 2F5 IgG1, with a 20 times lower IC99, a 10 times lower IC90 and a 33 times lower IC50 (IC99=10 nM and 200 nM, resp.; and IC90=3.3 nM and 33 nM, resp.). These differences were statistically significant, with p=0.005 for the IC99's, p=0.006 for IC90's, and p=0.004 for IC50's (FIG. 8).

Finally, the two 2F5 isotype neutralization activities were compared in the TZM-bl cell assay against the pseudovirus expressing HIV-1 QH0692.42 envelope (Bomsel, Tudor et al. 2011). In this assay again, 2F5 IgA2 IC90 and IC50 were 23.8 and 8.33 times lower, respectively, than those of the IgG1 one (Table 3, IC90=0.31 nM versus 7.38 nM, and IC50=0.024 nM versus 0.2 nM, resp).

Altogether, this set of functional tests demonstrated that 2F5 IgA2 and IgG1 have different antiviral activities in vitro. 2F5 IgA2 had a higher ability to block the HIV-1 transcytosis and infection of cells expressing CD4 and CCR5 receptors (CEM-CCR5+, and TZM-bl). 2F5 IgG1 had a slightly better potential to inhibit the virus transfer from LCs to CD4+ T lymphocytes. However, combining both isotypes resulted in a synergistic inhibition of virus transfer from LCs to autologous T cells and neutralization of CD4+ T lymphocyte infection.

### Identification of 2F5 IgA2 and IgG1-Specific Epitopes

Although both 2F5 isotypes recognized the 2F5 IgG1 canonical epitope ELDKWA, the observed differences in antiviral activities suggested that each antibody could recognize larger but different 3-dimensional epitopes. Phage display is widely used to analyze humoral immune responses and to map antibody epitopes. The inventors therefore used random peptide libraries expressed on M13 phages to determine comparatively the specific sequences able to bind each antibody isotype, and therefore corresponding to the antibody epitope, referred to as mimotope.

Using 2F5 IgG1 (resp. IgA2), 25 (resp. 22) different clones were isolated from the 12-mer random peptide library. All sequences without internal gaps (corresponding to ambiguously sequenced residues) were considered. In a first approach, to investigate the existence of a conserved motif in the isolated sequences, a multiple alignment was performed using the t_coffee server. Multiple alignment analyses of the selected sequences and the gp41 C-helix region are presented in FIGS. 9 and 10.

Sequences screened on 2F5 IgA2 shared a well-conserved motif centred around LDKWA (SEQ ID No 61). In contrast, for 2F5 IgG1, the consensus sequence is shorter (DKW) and deviates from 2F5 Ig A2 (only 5 sequences contain LDKWA and 2 LDRWA (SEQ ID No 62)). Moreover, three peptides only share few or no sequence identity with the reference (Peptides 1-6, 1-9 and 1-15). For both isotypes, termini parts of specific sequences retrieved by screening could not be mapped to the sole gp41 C-helix. This suggests that both antibodies recognize a conformational binding site apart from the GP-41 canonical motif ELDKWA. The inventors thus sought to identify their respective three-dimensional conformational epitopes using in silico approaches.

in Addition to the 2F5 IgG Epitope on the MPR, 2F5 IgG1 Bound to Another C-Helix Epitope Whereas IgA2 Bound to the N-Helix of gp41

Until recently, identification of conformational epitopes was hampered by difficulties in projecting the linear retrieved sequence onto a protein structure comprising the epitope. In the last years, softwares were developed that allow for three-dimensional (3D) analysis. These programs project the linear peptide sequence onto the 3D surface structure of target proteins by using published protein structure files. Several in silico approaches map 3D conformational epitopes on the surface of a protein structure (obtained by NMR or X-Ray) by using algorithms that take into account the relative distance and physicochemical properties of individual amino acids (see methods). A discontinuous 3D epitope is localized on the protein surface by searching for a 3D fit with partial amino acid strings of a given sequence in a pre-set distance on the protein surface. This algorithm is repeated for each string of amino acids until the full peptide sequence is analyzed. For each gp41 chain, the frequency of the residues predicted to belong to an epitope was plotted along the protein sequence.

Using this algorithm it was found that in addition to binding to ELDKWA (SEQ ID No 58), located on gp41 C helix, 2F5 IgG1 recognized a second but different motif, namely NYTSLIHSLI (SEQ ID No 59) on the same helix whereas IgA2 bound to the gp41 N-helix in the spatial vicinity of ELDKWA, defining a new conformational binding site on gp41 around the ASMTLTVQAR (SEQ ID No 60) cluster. The normalized Pepitope score allows to better evaluate the antibody binding efficiency to gp41. Strikingly, the score associated to the cluster NYTSLIHSLI recognized by 2F5 IgG1 (19.647) is much lower than those associated to the second cluster ASMTLTVQAR recognized by IgA2 (261.52). Mapped onto the gp41 protein structure, these results clearly showed that 2F5 IgG1 and IgA2 recognize a common and a specific motif that could potentially explain observed biological properties.

### Immunogenic Effect of Conformational Epitopes

11 of the 2F5IgA2 conformational epitopes and 8 of the 2F5IgG1 conformational epitopes identified were tested for immunogenicity in mice in a prime-boost regimen with recombinant gp41. Mice were primed intramucosally with recombinant gp41, followed by 5 intramucosal boosts with M13 phages expressing 11 2F5IgA2 epitopes or 8 2F5IgG1 epitopes.

The epitopes tested were SEQ ID NOs 2, 7, 8, 9, 10, 14, 17, 18, 20, 21 and 22 for 2F5IgA2, and SEQ ID NOs 25, 32, 34, 35, 36, 37, 39 and 40 for IgG1.

Serum was collected a day 75 and analyzed by standard techniques in ELISA against a recombinant gp41 protein.

The results are shown in Table 1. Boosting with either IgG1 or IgA2 epitopes gave substantially increased titers of anti-gp41 antibodies than were obtained by immunisation with recombinant gp41 alone (represented by control boost with M13 page only).

TABLE 1

| | Serum Day 75 | | Mice/group |
|---|---|---|---|
| 5 Boosts with | gp41 specific IgG | Mean titer | n = |
| M13 only | CT | <1000 | 5 |
| M13-8x IgG1 epitopes | 2F5γ + pCCL28 | 3 250 | 8 |
| M13-11x IgA2 epitopes | 2F5α − pCCL28 | 3000 | 4 |
| M13-11x IgA2 epitopes | 2F5α + pCCL28 | 10 250 | 8 |

### Discussion

To better evaluate the influence of antibody isotype on the protection against HIV-1 infection at the mucosal level, the inventors successfully produced and characterized a set of recombinant human IgG1 and IgA2 that carries the H and L chain variable domains of the broadly neutralizing HIV-1 gp41-specific Ab 2F5, and characterized their functional activities in vitro.

The study focused on the IgA2 isotype since it is less susceptible to degradation compared to IgA1, which hinge region represents a target for the mucosal IgA1 protease. In addition, 2F5 had already been switched to a dimeric IgA1 without improvement in the affinity and exhibited a limited HIV-1 neutralizing activity as compared with the IgG isoform (Wolbank, Kunert et al. 2003, J Virol 77(7): 4095-103). Furthermore, 2F5 dimeric-IgA1 was less potent than monomeric IgA2 in blocking HIV transcytosis across rectal tissue (Shen, Drelichman et al. 2010, J Immunol 184(7): 3648-55.).

Differences in the antigen binding of antibodies with identical VH/VL domains were generally assigned to the greater number of antigen binding sites, as it is the case for pentameric IgM and dimeric IgA antibodies (isotype-dependent avidity) compared to IgG. Avidity has usually a strong influence on antibody anti-viral activities. However, in the case of HIV-1, largely adept of evading antibody neutralization due to the low density of the envelope spike trimer at the viral surface, the avidity is not determinant. This is in contrast to non-enveloped viruses, for which the antibody avidity is important for the neutralization, due to their outer protein shell containing closely spaced epitopes.

Here, the avidity parameter is not involved in the differences between the IgG1 and IgA2 isoform of the HIV-1 gp41-specific Ab 2F5, because both isotypes are monomeric. Rather in all approaches used (FIGS. 1-4), the IgA2 isotype exhibits an enhanced affinity for gp41 subunits as compared to the IgG1: (i) binding to antigen-coated ELISA plates, (ii) competition ELISA between both isotypes for the cognate antigen binding (iii) ELISA with the antigen presented on liposomes-P1 in a lipid context and (iv) capture ELISA of free virus.

The results disclosed hereinprovide additional evidence that antibody fine specificity (referring here to differences in antigen binding by antibodies with the same specificity) can be a function of the isotype. The antibody affinity results from the process of affinity maturation carried out especially at the level of the complementary determining region (CDR) loops and, particularly for anti-HIV antibodies, at the level of VH domain framework loops. However, additional mechanisms could contribute to the functional affinity and specificity. Structural differences produced by the amino acids residues that are far from the antigen binding site, or that do not interact with the antigen, such as those from the CH1 domain, could generate electrostatic and hydrophobic interactions (pH, ionic strength . . . ) that change the molecular flexibility of the whole antibody and affect the antigen-binding site conformation.

These results suggest that the CH1 domain participates actively to the high binding affinity of 2F5 IgA2 for its target. The CH1 would allow the antigen-binding site to establish higher affinity interactions with the antigen. Accordingly, 2F5 IgA2 and IgG1 isotypes binding to gp41 was different. Molecular modeling showed that W666 and Lys665 from the ELDKWA epitope interact tightly with the 2F5 IgA2 paratope (FIG. 5), in agreement with the higher affinity of the IgA2 for ELDKWA, and P1 in ELISA. Furthermore, 2F5 IgA2 interacts not only with the expected 2F5 epitope (ELDKWA) located on gp41 C-helix, but also with an additional region in the spatial vicinity of ELDKWA on the adjacent gp41 N-helix (ASMTLTVQAR), defining a new conformational binding site on gp41. In contrast, 2F5 IgG1 interacts with a different epitope (NYTSLIHSLI) located on N-terminal of the C-helix, at a distance of around 30 nm, that most likely preclude binding of the two motifs by the two Fab sites of a single IgG1 molecule.

Furthermore, these differences in 3-D epitope specificity correlate with a lack of polyspecificity of 2F5 IgA2 (not shown), in contrast to 2F5 IgG1 which appears polyreactive, a property common among broadly neutralizing antibodies. The absence of polyreactivity might be also associated with the CH1 domain. The CH1 amino acids environment may contribute to fine-tuning the hydrophobic interactions leading to an increased affinity of 2F5 IgA2, expressed by the tight contact with the highly conserved residues (W666 and Lys665) in gp41MPER, and avoiding structural plasticity that mediate polyspecificity.

The higher affinity of IgA2 isotype for its target could be rationalized as follows, considering the sequence from the adjacent gp41 N-helix as being part of the 2F5 IgA2 epitope: either 2F5 IgA2 may first bind to the gp41 N-helix allowing antibody anchoring for subsequent conformational adjustment after this initial Ab-Ag encounter, thereby favoring the tighter interactions of IgA2 with W666 and the Lys665; or, alternatively, 2F5 IgA2 may first bind to the gp41 C-helix, and the strong interaction of IgA2 with the W666 and the Lys665 could consequently impose on the binding to the ASMTLTVQAR sequence that is situated in close spatial vicinity. In any case, the change in antibody structure upon binding to its antigen might be sufficient to account for changes in affinity related to stereo chemical significant movements of antibody side chains.

Although 2F5 IgA2 and IgG1 share the same VL and VH regions, their respective CH1 differs by 70% in sequence. The amount and distribution of electrostatic and hydrophobic interactions present within each CH1 domain modifies structural and/or kinetic constraints of the binding site, and in turn flexibility/rigidity of each paratope. Furthermore, the role of CH1 domain of IgA2 can be attributed to a change in the affinity of the antibody through a mechanism involving also a tighter interaction with ELDKWA as compared to IgG1 isotype as shown here by bioinformatics analyses, and at the cellular level. Binding of 2F5 IgA2 to the second novel motif on gp41 N-helix certainly involves the CH1 alpha domain as it is also part of the 3D epitope of two gp41 specific Fab IgA (not shown) with potent antiviral activities. Thus, differences in isotypes affinity correlate with their respective paratope conformation.

In addition to differences in the affinity of 2F5 IgA2 and IgG1 isotypes, the present results demonstrate the 3-dimensional complexity of the 2F5 epitope, adding up to earlier studies extending the nominal ELDKWA epitope to the linear sequence NEQELLELDKWASLWN (SEQ ID No 63). This 3-dimensional epitope differs between 2F5IgG1 and IgA2 isotypes and could account for the differences observed in their biological properties. Hence, the novel 3-dimensional epitope specific of 2F5 IgA2 we describe here includes a motif situated on the N-helix of gp41 but arranged in the spatial vicinity of ELDKWA. This 3D epitope is most likely exposed in the pre-fusion conformation of HIV envelope spike, namely before virus binding to epithelial cells or during transcytosis, a process that does not require a change in HIV envelope spike conformation. IgA2, but not IgG1 binding to that epitope, could result to the higher transcytosis blocking efficiency of IgA2.

In contrast, the additional epitope to ELDKWA recognized by 2F5 IgG1 on the N-terminal part of gp41 C-helix, namely NYTSLIHSLI, is more accessible on gp41 when the virus spike changes conformation to achieve fusion with CD4+ T cells (Chan and Kim 1998). As a consequence, 2F5 IgG1 is more efficient in neutralization of CD4+ cell infection, by either blocking binding to CD4+ T cells directly or following transfer from LCs, two mechanisms that engage a change in HIV envelope spike conformation with gp41 adopting fusion state allowing access in the N-terminal region of the C-helix of gp41. It may also account for the synergy of 2F5 IgA2 and IgG1: 2F5 IgA2 could bind better the virion before it interacts with the LCs (as also shown by virus capture ELISA, and ELISA with the antigen presented on liposomes-P1 in a lipid context, FIG. 4 and Table 2) whereas the 2F5 IgG1 could also bind gp41 in the fusion state using NYTSLIHSLI epitope that is better exposed. Consequently, the virus binding by both isotypes drive to an enhancement of the neutralization or to a completely block of the virus transfer.

Finally, the distance between gp41 monomers in the envelope spike is shorter than that between two variable regions of an IgG3 (the original isotype of 2F5 or IgA1, but similar to that of an IgA2 favouring its binding to gp41.

HIV-1 is mainly transmitted at mucosal sites through several pathways including transcytosis, uptake by dendritic cells, LCs, macrophages and intraepithelial CD4+ T cells or by penetrating through the gaps between epithelial cells. The inventors show here that 2F5 antibodies interfere with the initial steps of HIV-1 mucosal transmission. Both isotypes blocked HIV-1 transcytosis, reduced the infection of CD4+ T cells by CCR5-tropic, Glade B HIV-1 JR-CSF, and inhibited HIV-1 transfer from LCs to autologous CD4+ T cells. However, 2F5 IgA2 was more efficient in blocking the virus transcytosis, and inhibiting CD4+ cells infection, while 2F5 IgG1 was superior in blocking HIV-1 transfer from LCs to CD4+ T cells.

Of note, the inhibition of transcytosis is operational only in monostratified epithelium such as the endocervix and the rectum, but not in pluristratified epithelium found in foreskin and vagina that are most vulnerable to HIV. The higher ability of 2F5 IgA2 to block CD4+ cell infection suggests that 2F5 IgA2 could also protect multilayered squamous epithelia from HIV-1 entry, whereas IgG1 2F5 could reduce the transfer of virus from LCs to T cells.

Antibody-dependent cell mediated cytotoxicity (ADCC) of HIV infected cells by IgG1 and IgA antibodies from cervicovaginal fluids and colostrums of HIV-infected women could also participate in local protection to HIV-1. Accordingly, we have recently reported that 2F5 IgG1 has an in vitro potential to mediate the ADCC of both X4-tropic HIV-1 RI dependent-envelope-expressing cells, and R5-HIV infected cells in a Fc manner (Tudor and Bomsel, 2011, Aids 25(6): 751-9). In addition, we have recently shown that in gp41-vaccinated monkey challenged by the vaginal route with SHIV that remain uninfected despite 13 low dose challenges, one correlate to protection was the presence of mucosal IgG specific for gp41MPER with in vitro ADCC activities (Bomsel, Tudor et al 2011, Immunity 34(2): 269-80).

2F5 IgA2 antibody also triggered ADCC of HIV-1 envelope gp41 subunit coated cells and monocytes as effector cells but with a lower efficacy than 2F5 IgG1 (not shown), most likely because it is mainly IgA1 that can trigger ADCC (Black, Cummins et al. 1996) due to its higher hinge region flexibility allowing for better dual binding to antigen and Fc receptors.

Finally, the synergy of 2F5IgA2 and IgG1 in blocking HIV-1 transfer from LCs to autologous T cells, and in reducing CD4+ T cell infection suggests that both isotypes could participate together in protection of the vaginal pluristratified epithelia prior to virus dissemination.

In conclusion, the present results provide novel evidence in favour of the dominant role of the antibody CH1 domain in defining the affinity of the 2F5 antibody for its antigen gp41. The participation of CH1 in antigen binding can further explain the differences in the anti-viral activity of 2F5 IgA2 compared to IgG1. Furthermore, the present results show that both isotypes IgG1 and IgA2 of 2F5 Ab interfere in vitro with the earliest steps of HIV-1 transmission across mucosal surfaces and suggest that in vivo IgA and IgG antibodies could have complementary and synergistic anti-viral activities.

TABLE 2
The affinity of 2F5-IgA for gp41 MPER is higher than that
of 2F5 IgG1

KD nM

| | | 2F5 IgA2 | 2F5 IgG1 |
|---|---|---|---|
| P1: Lipids | V1 | 0.024 | 0.31 |
| 1 for 100 | R1 | 0.05 | 0.50 |
| | NR1 | 0.21 | 0.69 |
| P1: Lipids | V2 | 0.024 | 0.41 |
| 1 for 20 | R2 | 0.061 | 0.51 |
| | NR2 | 0.15 | 1.33 |
| No lipid | P1 | 0.2 | 7.38 |

Binding affinities of 2F5Abs to various liposomes P1 were measured by ELISA as described in Materials and Methods.
V = viral liposomes,
R = raft liposomes,
N = non-raft liposomes.

TABLE 3
2F5 IgA2 has a higher potential than 2F5 IgG1 to neutralize
TZM-bl cell infection by R5 tropic HIV-1.
Neutralization of envelope pseudotyped HIV-1 R5 tropic
QH0692.42 infection mediated by 2F5 Abs was evaluated
using the TZM-bl assay. The antibody concentrations causing
50% (IC50) and 90% (IC90) reductions in luciferase reporter
gene production were determined by regression analysis.

Abs
concentration
(nM)

| Abs | IC50% | IC90% |
|---|---|---|
| 2F5 IgA2 | 0.024 | 0.31 |
| 2F5 IgG1 | 0.2 | 7.38 |

1. An isolated peptide comprising or consisting of the consecutive sequence of amino acids of a sequence shown in
(i) FIG. 9 (SEQ ID Nos 1-22); or
(ii) FIG. 10 (SEQ ID Nos 23-47).

2. A vaccine composition comprising an agent selected from the group consisting of (i) one or more peptides according to claim 1, and (ii) a nucleic acid encoding said peptide.

3. A vaccine composition according to claim 2, which composition comprises an agent selected from the group consisting of (i) at least one peptide of FIG. 9 (SEQ ID Nos 1-22), and (i) a nucleic acid encoding said peptide.

4. A vaccine composition according to claim 3, which composition further comprises an agent selected from the group consisting of (i) at least one peptide of FIG. 10 (SEQ ID Nos 23-47) and (ii) a nucleic acid encoding said peptide.

5. A vaccine composition according to claim 4, which composition comprises an agent selected from the group consisting of (i) a peptide of one of more of SEQ ID Nos 8, 9, 10, 32, 34, 35, 36, 37 and 40, and (ii) a nucleic acid encoding said peptide.

6. A vaccine composition according to claim 1, which is suitable for mucosal administration.

7. A vaccine composition according to claim 1, further comprising an adjuvant.

8. A method of preventive treatment of HIV infection or AIDS comprising administering to a subject a therapeutically effective amount of an agent selected from the group consisting of (i) a peptide of claim 1, and (ii) a nucleic acid encoding said peptide.

9. A method of inducing a mucosal immune response to HIV-1 comprising administering to a subject a therapeutically effective amount of an agent selected from the group consisting of (i) a peptide of claim 1, and (ii) a nucleic acid encoding said peptide.

10. A method of inducing a mucosal IgA2 response to HIV-1 comprising administering to a subject an agent selected from the group consisting of (i) a peptide according to claim 1, and (ii) a nucleic acid encoding said peptide, wherein said peptide has a sequence of FIG. 9 (SEQ ID Nos 1-22).

11. A method of inducing a mucosal IgG1 response to HIV-1 comprising administering to a subject an agent selected from the group consisting of (i) a peptide according to claim 1, and (ii) a nucleic acid encoding said peptide, wherein said peptide has a sequence of FIG. 10 (SEQ ID Nos 23-47).

12. An IgA2 antibody having a heavy chain comprising one or more of the following CDR sequences:
VH CDR1
(SEQ ID No 50)
GFSLSDFGVG;
VH CDR2
(SEQ ID No 51)
IYSDDDK;
and
VH CDR3
(SEQ ID No 52)
AHRRGPTTLFGVPIARGPVNAMDV;
and/or a light chain comprising one or more of the following CDR sequences
VL CDR1
(SEQ ID No 53)
QGVTSA;
VL CDR2
(SEQ ID No 54)
DAS;
and
VL CDR3
(SEQ ID No 55)
QQLHFYPHT;
and optionally having a heavy chain comprising the amino acid sequence:
(SEQ ID No 56)
RITLKESGP.PLVKPTQTLTLTCSFSGFSLSDFGVGVGWIRQPPGKALE
WLAIIYSDDDKRYSPSLN.TRLTITKDTSKNQVVLVMTRVSPVDTATYF
CAHRRGPTTLFGVPIARGPVNAMDVWGQGITVTI
and/or a light chain comprising the amino acid sequence:
(SEQ ID No 57)
ALQLTQSPSSLSASVGDRITITCRASQGVTSALAWYRQKPGSPPQLLIY
DASSLESGVPSRFSGSGSGTEFTLTISTLRPEDFATYYCQQLHFYPHTF
GGGTRVDVR.

13. The antibody of claim 12 for use in the preventive treatment of HIV infection or AIDS.

14. A method of diagnosis of HIV infection or AIDS, said method comprising detection of a gp41 protein in a biological sample from a patient using an antibody according to claim 12.

15. An isolated nucleic acid encoding the amino acid sequence of a peptide according to claim 1.

16. A method of prevention or treatment of HIV infection, comprising administration of a vaccine composition of claim 2 to an individual in need thereof.

17. A vaccine composition according to claim 2, which is suitable for mucosal administration.

18. A vaccine composition according to claim 3, which is suitable for mucosal administration.

19. A vaccine composition according to claim 4, which is suitable for mucosal administration.

20. A vaccine composition according to claim 2, which is suitable for mucosal administration.


SEQUENCE LISTING (63 sequences)

SEQ ID NO: 1 - protein, 12 aa, Human immunodeficiency virus
QHQTWDKWSHLG

SEQ ID NO: 2 - protein, 12 aa, Human immunodeficiency virus
SGTLDKWTSLPR

SEQ ID NO: 3 - protein, 12 aa, Human immunodeficiency virus
TLTVDKWATLRA

SEQ ID NO: 4 - protein, 12 aa, Human immunodeficiency virus
HAQPLDKWAYLV

SEQ ID NO: 5 - protein, 12 aa, Human immunodeficiency virus
QQSFIPDKWHDL

SEQ ID NO: 6 - protein, 12 aa, Human immunodeficiency virus
STRYALDKWSHI

SEQ ID NO: 7 - protein, 12 aa, Human immunodeficiency virus
QSSNWDKWAILP

SEQ ID NO: 8 - protein, 12 aa, Human immunodeficiency virus
TMDQLDKWAPLE

SEQ ID NO: 9 - protein, 12 aa, Human immunodeficiency virus
ELDRWADLPRYL

SEQ ID NO: 10 - protein, 12 aa, Human immunodeficiency virus
TPDKWSYLSNLQ

SEQ ID NO: 11 - protein, 12 aa, Human immunodeficiency virus
KHGLTLDRWASL

SEQ ID NO: 12 - protein, 12 aa, Human immunodeficiency virus
ESGSHDKWADLS

SEQ ID NO: 13 - protein, 12 aa, Human immunodeficiency virus
NMSYDKWYNLPT

SEQ ID NO: 14 - protein, 12 aa, Human immunodeficiency virus
HDVLDKWATLKE

SEQ ID NO: 15 - protein, 12 aa, Human immunodeficiency virus
SQAEFDKWAEVR

SEQ ID NO: 16 - protein, 12 aa, Human immunodeficiency virus
THSLPLDKWAIV

SEQ ID NO: 17 - protein, 12 aa, Human immunodeficiency virus
APQMEQDKWTMI

SEQ ID NO: 18 - protein, 12 aa, Human immunodeficiency virus
ELFQDADKWHSL

SEQ ID NO: 19 - protein, 12 aa, Human immunodeficiency virus
GKTNAWDKWAFL

SEQ ID NO: 20 - protein, 12 aa, Human immunodeficiency virus
WTLSELDKWATL

SEQ ID NO: 21 - protein, 12 aa, Human immunodeficiency virus
MIPDKWNAMRVG

SEQ ID NO: 22 - protein, 12 aa, Human immunodeficiency virus
AADPDKWVNLPP

SEQ ID NO: 23 - protein, 12 aa, Human immunodeficiency virus
YAPDKWQNLLPY

SEQ ID NO: 24 - protein, 12 aa, Human immunodeficiency virus
QQSFIPDKWHDL

SEQ ID NO: 25 - protein, 11 aa, Human immunodeficiency virus
TSLDKWAMKMP

SEQ ID NO: 26 - protein, 12 aa, Human immunodeficiency virus
AKQTLDKWALFG

SEQ ID NO: 27 - protein, 12 aa, Human immunodeficiency virus
APQMEQDKWTMI

SEQ ID NO: 28 - protein, 12 aa, Human immunodeficiency virus
NAEDKWAFLHVA

SEQ ID NO: 29 - protein, 12 aa, Human immunodeficiency virus
SFDYWPVPDRTR

SEQ ID NO: 30 - protein, 12 aa, Human immunodeficiency virus
QAMDKWSLLGTR

SEQ ID NO: 31 - protein, 12 aa, Human immunodeficiency virus
YMDKWANIVALQ

SEQ ID NO: 32 - protein, 12 aa, Human immunodeficiency virus
TMDQLDKWAPLE

SEQ ID NO: 33 - protein, 11 aa, Human immunodeficiency virus
TGPFTNISSLH

SEQ ID NO: 34 - protein, 12 aa, Human immunodeficiency virus
ELDRWADLPRYL

SEQ ID NO: 35 - protein, 12 aa, Human immunodeficiency virus
AYDEVDRWAFLD

SEQ ID NO: 36 - protein, 11 aa, Human immunodeficiency virus
QSLDKWAMFMP

SEQ ID NO: 37 - protein, 12 aa, Human immunodeficiency virus
NMSYDKWYNLPT

SEQ ID NO: 38 - protein, 12 aa, Human immunodeficiency virus
NMMDKWYMVTSG

SEQ ID NO: 39 - protein, 12 aa, Human immunodeficiency virus
QSSNWDKWAILP

SEQ ID NO: 40 - protein, 12 aa, Human immunodeficiency virus
GDELDRWAWMPT

SEQ ID NO: 41 - protein, 12 aa, Human immunodeficiency virus
WTLSELDKWATL

SEQ ID NO: 42 - protein, 12 aa, Human immunodeficiency virus
ELQKDRWASLFL

SEQ ID NO: 43 - protein, 12 aa, Human immunodeficiency virus
LIPDKWSSIFAM

SEQ ID NO: 44 - protein, 11 aa, Human immunodeficiency virus
ALVSDRWSWMH

SEQ ID NO: 45 - protein, 12 aa, Human immunodeficiency virus
VKKDQTAVYNHY

SEQ ID NO: 46 - protein, 12 aa, Human immunodeficiency virus
ELFQDADKWHSL

SEQ ID NO: 47 - protein, 12 aa, Human immunodeficiency virus
TLDKWSNLFTFN

SEQ ID NO: 48 - protein, 58 aa, Human immunodeficiency virus
DDDDKMEWDREYNNYTSLIHSLIEESQNQQEKNEQELLELDKWASLWNWFNNWLWYIT

SEQ ID NO: 49 - protein, 51 aa, Human immunodeficiency virus
GAASMTLTVQARQLLSGIVQQQNNLLRAIEAQQHLLQLTVWGIKQLQARIL

SEQ ID NO: 50 - protein, 10 aa, Homo sapiens
GFSLSDFGVG

SEQ ID NO: 51 - protein, 7 aa, Homo sapiens
IYSDDDK

SEQ ID NO: 52 - protein, 24 aa, Homo sapiens
AHRRGPTTLFGVPIARGPVNAMDV

SEQ ID NO: 53 - protein, 6 aa, Homo sapiens
QGVTSA

SEQ ID NO: 54 - protein, 3 aa, Homo sapiens
DAS

SEQ ID NO: 55 - protein, 9 aa, Homo sapiens
QQLHFYPHT

SEQ ID NO: 56 - protein, 130 aa, Homo sapiens
RITLKESGPPLVKPTQTLTLTCSFSGFSLSDFGVGVGWIRQPPGKALEWLAIIYSDDDKRYSPSLNTRLTITKDTSKNQVVLVMTRVSPVDTATYFCAHRRGPTTLFGVPIARGPVNAMDVWGQGITVTI

SEQ ID NO: 57 - protein, 107 aa, Homo sapiens
ALQLTQSPSSLSASVGDRITITCRASQGVTSALAWYRQKPGSPPQLLIYDASSLESGVPSRFSGSGSGTEFTLTISTLRPEDFATYYCQQLHFYPHTFGGGTRVDVR

SEQ ID NO: 58 - protein, 6 aa, Human immunodeficiency virus
ELDKWA

SEQ ID NO: 59 - protein, 10 aa, Human immunodeficiency virus
NYTSLIHSLI

SEQ ID NO: 60 - protein, 10 aa, Human immunodeficiency virus
ASMTLTVQAR

SEQ ID NO: 61 - protein, 5 aa, Human immunodeficiency virus
LDKWA

SEQ ID NO: 62 - protein, 5 aa, Human immunodeficiency virus
LDRWA

SEQ ID NO: 63 - protein, 16 aa, Human immunodeficiency virus
NEQELLELDKWASLWN